(19) United States
(12) Patent Application Publication
(10) Pub. No.: US 2013/0011833 A1
(43) Pub. Date: Jan. 10, 2013

(54) METHOD FOR IDENTIFYING NUCLEIC ACIDS BOUND TO AN ANALYTE

(75) Inventors: Stephen R. Quake, Singapore (SG); William F. Burkholder, Singapore (SG); Tze Howe Charn, Singapore (SG)

(73) Assignee: Agency for Science, Technology and Research, Connexis (SG)

(21) Appl. No.: 13/457,288

(22) Filed: Apr. 26, 2012

Related U.S. Application Data
(60) Provisional application No. 61/479,236, filed on Apr. 26, 2011.

(51) Int. Cl. C12Q 1/68

(52) U.S. Cl. 435/6.11; 435/6.12

(57) ABSTRACT

A method for identifying a target nucleic acid bound by an analyte in a sample comprising: (a) contacting said sample with a first probe comprising a first nucleic acid and a first analyte binding domain and a second probe comprising a second nucleic acid and second analyte binding domain, wherein said first and second probes can bind to said analyte, such that said first and second nucleic acid are in spatial proximity to form a complex with said target nucleic acid if said target nucleic acid is bound by said analyte in said sample; (b) incubating said sample with a ligase that can ligate said complex to form a ligated target nucleic acid template; (c) amplifying said target nucleic acid template if present in said sample, and (d) detecting the presence or absence of an amplified target nucleic acid template.

### CROSS-REFERENCE TO RELATED APPLICATIONS

This application claims the benefit of priority of U.S. provisional application No. 61/479,236, filed Apr. 26, 2011, the contents of which is hereby incorporated by reference in its entirety for all purposes.

### FIELD OF THE INVENTION

The present invention relates in general to the field of molecular biology, including methods for chromatin characterization. In some embodiments, the present invention relates to a method for identifying or quantifying one or more target nucleic acids bound by one or more analytes.

### BACKGROUND OF THE INVENTION

Protein-DNA interactions are primarily responsible for the regulation of gene expression in cells. The misregulation of protein-DNA interactions often leads to human diseases. A key challenge has been to determine the occupancy of transcription factors on chromatin in an unbiased, sensitive and high throughput fashion. Chromatin immunoprecipitation (ChIP) is a powerful technique to detect protein-DNA interactions in vivo. Nonetheless, current ChIP protocols require a mechanical method for physically separating antibody-bound protein-DNA complexes from DNA, such as centrifugation, collection in a chromatography column or magnetic capture, followed by repeated washing to dilute out unbound material and remove non-specifically bound proteins and DNA. These mechanical steps are time-consuming, difficult to automate, and among the greatest sources of variations in signal strength and signal-to-noise due to losses of material and uncontrolled variability in the washing steps. In addition, ChIP tends to require large amounts of material; conventional protocols require millions of cells, and much effort has been invested in finding elaborate protocols to reduce the number of cells.

The ability to assay in vivo protein binding and the distribution of epigenetic marks across the genome starting with many fewer cells would greatly facilitate experiments in many areas where sample material is limiting, such as in developmental, stem cell, and cancer biology, immunology, and medical applications with patient samples.

Proximity ligation was first developed as a sensitive method for detecting and quantitating the abundance of specific antigens such as proteins, viruses, and cells. In this assay, a unique DNA tag signaling the presence of the antigen is generated when two distinct DNA molecules (half-tags) are ligated together. Generation of the ligation product is made to depend on the presence of the antigen by linking each half-tag to an antibody or aptamer that recognizes the antigen. In the presence of the antigen, two antibodies carrying different half-tags can be brought together in the same ternary complex. If the samples are sufficiently dilute, when DNA ligase is added, the rate at which ligation occurs within antigen-dependent ternary complexes is significantly faster than the background rate of ligation between freely diffusing half-tags due to the higher local concentration.

The limitations of current techniques of chromatin analysis, including chromatin immunoprecipitation, are well known. In particular, they are cumbersome, insensitive, subject to operator variability, and are challenging to reproduce. Additionally current chromatin analysis techniques are not well suited to scaling up or multiplexing for the detection of multiple analyte targets.

Whilst refinements to the basic protocol for chromatin immunoprecipitation have been described for increasing its sensitivity and dynamic range, no improvements have previously been made that eliminate the need for mechanically separating antibody-bound protein-DNA complexes from DNA.

### SUMMARY OF THE INVENTION

In a first aspect there is provided a method for identifying a target nucleic acid bound by an analyte in a sample comprising: contacting said sample with a first probe comprising a first nucleic acid or first oligonucleotide (oligo) tag and a first analyte binding domain and a second probe comprising a second nucleic acid or second oligo tag and second analyte binding domain, wherein said first and second probes can bind to said analyte, such that said first and second nucleic acid or first and second oligo tag are in spatial proximity to form a complex with said target nucleic acid if said target nucleic acid is bound by said analyte in said sample; incubating said sample with a ligase that can ligate said complex to form a ligated nucleic acid template; amplifying said target nucleic acid template if present in said sample; detecting the presence or absence of an amplified nucleic acid template.

In a second aspect there is provided a method for identifying a target nucleic acid bound by an analyte in a sample comprising: contacting said sample with a first probe comprising a first and second nucleic acid or first and second oligo tag and a first analyte binding domain; and a second probe comprising a third and fourth nucleic acid or third and fourth oligo tag and a second analyte binding domain, wherein said second nucleic acid or second oligo tag provides a region of complementarity for said third nucleic acid or third oligo tag and one end of said target nucleic acid and said fourth nucleic acid or fourth oligo tag provides a region of complementarity for said first nucleic acid or first oligo tag and another end of said target nucleic acid, wherein said first and second probes can bind to said analyte, such that said first and second nucleic acids or first and second oligo tags and said third and fourth nucleic acids or third and fourth oligo tags are in spatial proximity to form a complex with said target nucleic acid if said target nucleic acid is bound by said analyte in said sample; incubating said sample with a ligase that can ligate said complex to form a ligated nucleic acid template; amplifying said target nucleic acid template if present in said sample; detecting the presence or absence of an amplified nucleic acid template.

In a third aspect there is provided a kit for determining binding of an analyte to a target nucleic acid in a sample comprising: a first container comprising a first probe, wherein said first probe comprises a first nucleic acid or first oligo tag and a first analyte binding domain; a second container comprising a second probe, wherein said second probe comprises a second nucleic acid or second oligo tag and second analyte binding domain; and instructions for using the first and second nucleic acid probes to detect an analyte in a sample.

In a fourth aspect, there is provided a kit for determining binding of an analyte to a target nucleic acid in a sample comprising: a first container comprising a first probe comprising a first and second nucleic acid or first and second oligo tag and a first analyte binding domain; a second container comprising a second probe comprising a third and fourth nucleic acid or third and fourth oligo tag and a second analyte binding domain; and instructions for using the first and second nucleic acid probes to detect an analyte in a sample.

The invention described herein details an approach which enables sensitive, fast, scalable, and convenient detection of chromatin state. Several new inventive steps are required in order to use proximity ligation to identify DNA sites in a genome or in a complex library of nucleic acids that are associated with analytes, specific proteins or covalent modifications.

Further disclosed herein are methods for detecting and quantifying an analyte-bound nucleic acid. Generally, the method comprises: (a) contacting a sample comprising an analyte and a nucleic acid molecule, which may be part of a sample containing a mixture of nucleic acid molecules, with a first probe comprising a first analyte binding domain and a first oligo tag; (b) attaching the first oligo tag to a first analyte-bound nucleic acid, wherein attachment of the oligo tag to the analyte-bound nucleic acid forms a probe-nucleic acid complex; (c) amplifying the probe-nucleic acid complex to form an amplified probe-nucleic acid product; and (d) detecting the amplified probe-nucleic acid product. The oligo tag may further comprise a linker compatible region. The oligo tag may further comprise a unique identification sequence. The oligo tag may further comprise a primer binding sequence. The method may further comprise contacting the sample with a second probe comprising a second analyte binding domain and a second oligo tag. The first probe may further comprise a third oligo tag. The second probe may further comprise a fourth oligo tag. Each probe may comprise two or more oligo tags. The analyte binding domain may be an antibody. The attaching step may comprise ligating the oligo tag to the analyte-bound nucleic acid. The method may further comprise attaching a linker to the analyte-bound nucleic acid. The attaching step may comprise ligating the oligo tag to the linker. The method may further comprise attaching a nucleic acid to an analyte to form an analyte-bound nucleic acid. The nucleic acid may be attached to the analyte by cross-linking. The analyte binding domain of the probe may be directly attached to the analyte-bound nucleic acid. Alternatively, the analyte binding domain of the probe may be indirectly attached to the analyte-bound nucleic acid. The method may further comprise contacting the sample with at least 2, 3, 4, 5, 10, 20, 30, 40, 50, 100, 200, 300, 400, 500, 600, 700, 800, 900, 1000 probes. The analyte binding domains of the probes may bind to at least 2, 3, 4, 5, 10, 20, 30, 40, 50, 100, 200, 300, 400, 500, 600, 700, 800, 900, 1000 analytes. The oligo tags of the probes may attach to at least 2, 3, 4, 5, 10, 20, 30, 40, 50, 100, 200, 300, 400, 500, 600, 700, 800, 900, 1000 analyte-bound nucleic acids. The analyte binding domain of the probes may bind to the same analyte. The analyte binding domain of the probes may bind to different analytes. The oligo tags of the probes may attach to the same analyte-bound nucleic acid. The oligo tags of the probes may attach to different analyte-bound nucleic acids. The method may further comprise heating the sample. The method may further comprise incubating the sample in a high salt solution. The method may further comprise cleaving the oligo tag from the probe. The method may further comprise quantifying the amplified probe-nucleic acid product. The method may further comprise sequencing the amplified probe-nucleic acid product. The method may further comprise attaching the amplified probe-nucleic acid product to a solid support. The solid support may comprise an array or bead.

Alternatively, methods for identifying an analyte-bound nucleic acid comprise: (a) contacting a sample with a first probe comprising a first oligo tag and a first analyte binding domain and a second probe comprising a second oligo tag and second analyte binding domain, wherein said first and second probes can bind to a first and second analyte, respectively, such that said first and second oligo tags are in spatial proximity to form a complex with a nucleic acid bound by said analyte in said sample; (b) attaching said first and second oligo tags to said nucleic acid to form a nucleic acid template; (c) amplifying said nucleic acid template; and (d) detecting the presence or absence of an amplified nucleic acid template.

Further disclosed herein are methods for identifying a analyte-bound nucleic acid in a sample comprising: (a) contacting said sample with a first probe comprising a first and second oligo tag and a first analyte binding domain; and a second probe comprising a third and fourth oligo tag and a second analyte binding domain, wherein said second oligo tag provides a region of complementarity for said third oligo tag and one end of said target nucleic acid and said fourth oligo tag provides a region of complementarity for said first oligo tag and another end of said target nucleic acid, wherein said first and second probes can bind to said analyte, such that said first and second oligo tags and said third and fourth oligo tags are in spatial proximity to form a complex with said target nucleic acid if said target nucleic acid is bound by said analyte in said sample; incubating said sample with a ligase that can ligate said complex to form a ligated nucleic acid template; amplifying said target nucleic acid template; and detecting the presence or absence of an amplified nucleic acid template.

In some embodiments, provided herein are methods for identifying an analyte-bound nucleic acid comprise: (a) contacting a sample with a first probe comprising a first oligo tag and a first analyte binding domain and a second probe comprising a second oligo tag and second analyte binding domain, wherein said first and second probes bind to a first and second analyte, respectively, such that said first and second oligo tags are in spatial proximity to form a complex with a nucleic acid bound by said analyte in said sample; (b) attaching said first and second oligo tags to said nucleic acid to form a nucleic acid template; (c) amplifying said nucleic acid template; and (d) detecting the presence or absence of an amplified nucleic acid template.

In other embodiments, provided herein are methods for identifying a analyte-bound nucleic acid in a sample comprising: (a) contacting said sample with a first probe comprising a first and second oligo tag and a first analyte binding domain; and a second probe comprising a third and fourth oligo tag and a second analyte binding domain, wherein said second oligo tag provides a region of complementarity for said third oligo tag and one end of said target nucleic acid and said fourth oligo tag provides a region of complementarity for said first oligo tag and another end of said target nucleic acid, wherein said first and second probes bind to said analyte, such that said first and second oligo tags and said third and fourth oligo tags are in spatial proximity to form a complex with said target nucleic acid if said target nucleic acid is bound by said analyte in said sample; incubating said sample with a ligase that ligates said complex to form a ligated nucleic acid template; amplifying said target nucleic acid template; and detecting the presence or absence of an amplified nucleic acid template.

Also disclosed herein are kits and compositions for identifying analyte-bound nucleic acids. Disclosed herein is a probe comprising: (a) an analyte binding domain; and (b) an oligo tag. The oligo tag may comprise a linker compatible region, unique identification sequence, a primer binding site, or any combination thereof. The analyte binding domain may be an antibody.

Further disclosed herein is a kit comprising: a plurality of probes, wherein the probe comprises an analyte binding domain and an oligo tag. The oligo tag may comprise a linker compatible region, unique identification sequence, a primer binding site, or any combination thereof. The analyte binding domain may be an antibody. The kits disclosed herein may further comprise a plurality of linkers. Additionally, the kits may include a ligating agent. In other cases, the kits may comprise instructions for attaching the probe to an analyte-bound nucleic acid.

In some embodiments, the kits and compositions disclosed herein may comprise a probe comprising (a) an analyte binding domain; and (b) an oligo tag comprising a linker compatible region, wherein said linker compatible region facilitates attachment of the probe to a nucleic acid. In other embodiments, the kits and compositions disclosed herein may comprise a probe comprising (a) an analyte binding domain; and (b) an oligo tag comprising a unique identification sequence, wherein said unique identification sequence confers a unique identifiable marker to said probe. The kits and compositions disclosed herein may comprise: (a) a first container comprising a first probe, wherein said first probe comprises a first oligo tag and a first analyte binding domain; a second container comprising a second probe, wherein said second probe comprises a second oligo tag and second analyte binding domain; and (b) instructions for using the first and second probes to detect an analyte-bound nucleic acid in a sample.

Further disclosed herein are kits and compositions for identifying analyte-bound nucleic acids in a sample comprising: (a) a first container comprising a first probe comprising a first and second oligo tag and a first analyte binding domain; a second container comprising a second probe comprising a third and fourth oligo tag and a second analyte binding domain; and (b) instructions for using the first and second probes to detect an analyte-bound nucleic acid in a sample.

### BRIEF DESCRIPTION OF THE DRAWINGS

The invention will be better understood with reference to the detailed description when considered in conjunction with the non-limiting examples and the accompanying drawings, in which:

FIG. 1 is a schematic of an embodiment of the disclosure as described herein.

FIG. 2A and FIG. 2B show the results of an analysis of the DNA binding specificity of estrogen receptor α (ERα) obtained using an embodiment of the method described herein.

FIG. 3 shows the results of an analysis of the DNA binding specificity of ERα obtained using an embodiment of the method described herein using deoxyadenine (dA)-tailed chromatin.

FIG. 4 shows the results of an analysis of the DNA binding specificity of ERα obtained using an embodiment of the method described herein using a 10-fold reduction in chromatin compared to the results obtained in FIG. 2, corresponding to the chromatin content of 5,000 cells.

FIG. 5 is a schematic of an embodiment of the disclosure as described herein.

FIG. 6 is a schematic of an embodiment of the disclosure as described herein.

### DETAILED DESCRIPTION OF THE PRESENT INVENTION

In one aspect of the invention, provided herein is a method for identifying a target nucleic acid bound by an analyte in a sample comprising: contacting said sample with a first probe comprising a first nucleic acid and a first analyte binding domain and a second probe comprising a second nucleic acid and second analyte binding domain, wherein said first and second probes can bind to said analyte, such that said first and second nucleic acid are in spatial proximity to form a complex with said target nucleic acid if said target nucleic acid is bound by said analyte in said sample; incubating said sample with a ligase can ligate said complex to form a ligated nucleic acid template; amplifying said target nucleic acid template; detecting the presence or absence of an amplified nucleic acid template.

In a second aspect of the invention, provided herein is a method for identifying a target nucleic acid bound by an analyte in a sample comprising: contacting said sample with a first probe comprising a first and second nucleic acid and a first analyte binding domain; and a second probe comprising a third and fourth nucleic acid and a second analyte binding domain, wherein said second nucleic acid provides a region of complementarity for said third nucleic acid and one end of said target nucleic acid and said fourth nucleic acid provides a region of complementarity for said first nucleic acid and another end of said target nucleic acid, wherein said first and second probes can bind to said analyte, such that said first and second nucleic acids and said third and fourth nucleic acids are in spatial proximity to form a complex with said target nucleic acid if said target nucleic acid is bound by said analyte in said sample; incubating said sample with a ligase that can ligate said complex to form a ligated nucleic acid template; amplifying said target nucleic acid template; detecting the presence or absence of an amplified nucleic acid template.

In one embodiment of the second aspect, the second and fourth nucleic acids provide a “bridge” or a “splint” between the ends of the target nucleic acid and the first and fourth nucleic acids. This permits localization of the ends of the first and third nucleic acids adjacent to the respective ends of the target nucleic acid for ligation. The numbering of the nucleic acids in the probes is interchangeable and is not intended to be limiting. Also, the location of the nucleic acids on the probes can be changed. Accordingly, in one embodiment of the second aspect, the first and third nucleic acids may be located on said first probe and said second and fourth nucleic acids may be located on said second probe. In one embodiment, one of the probes may comprise a first and second “bridge” nucleic acid.

In another embodiment, the first probe may comprise a first probe comprising a first nucleic acid and a first analyte binding domain and a second probe comprising a second nucleic acid and second analyte binding domain, wherein said first nucleic acid provides a region of complementarity for said second nucleic acid and an end of the target nucleic acid.

In one embodiment, the method further comprises the step of cross-linking the analyte to the target nucleic acid in said sample with an agent that can cross-linking said analyte to said target nucleic acid prior to contacting the analyte with said first and second probes. In one embodiment, the cross-linking agent is formaldehyde. It will be appreciated by those of skill in the art that other suitable cross-linking methods may also be used, for example pulsed laser UV cross-linking; the sequential treatment of cells with a bifunctional protein-protein cross-linking agent such as dimethyl adipimidate, dimethyl pimelimidate, ethylene glycol bis[succinimidylsuccinate], or disuccinimidyl glutarate followed by treatment with formaldehyde.

Disclosed herein are methods for detecting an analyte-bound nucleic acid. The analyte-bound nucleic acid can comprise some or all of the sequence bound by the analyte. In some embodiments the analyte-bound nucleic acid comprises some sequence adjacent to the binding site.

Generally, the method comprises: (a) contacting a sample comprising a protein and a nucleic acid molecule, which may be part of a sample containing a mixture of nucleic acid molecules, with a first probe comprising a first analyte binding domain and a first oligo tag and a second probe comprising an second analyte binding domain and a second oligo tag; (b) attaching the first oligo tag to a first analyte-bound nucleic acid and/or attaching the second oligo tag to a second analyte-bound nucleic acid, wherein attachment of the oligo tag to the analyte-bound nucleic acid forms a probe-nucleic acid complex; (c) amplifying the probe-nucleic acid complex to form an amplified probe-nucleic acid product; and (d) detecting the amplified probe-nucleic acid product. The oligo tag may further comprise a linker compatible region. The oligo tag may further comprise a unique identification sequence. The oligo tag may further comprise a primer binding sequence. The analyte binding domain may be an antibody. The attaching step may comprise ligating the first oligo tag and/or second oligo tag to the analyte-bound nucleic acid. The method may further comprise attaching a linker to the analyte-bound nucleic acid. The attaching step may comprise ligating the first oligo tag and/or second oligo tag to the linker. The oligo tag may be directly attached to the analyte-bound nucleic acid. Alternatively, the oligo tag may be indirectly attached to the analyte-bound nucleic acid. The method may further comprise attaching a nucleic acid to an analyte to form an analyte-bound nucleic acid. The nucleic acid may be attached to the analyte by cross-linking.

In some embodiments the method does not include a second probe. The method comprises: (a) contacting a sample comprising a protein and a nucleic acid molecule with a probe comprising an analyte binding domain and a first oligo tag; (b) attaching the oligo tag to a analyte-bound nucleic acid wherein attachment of the oligo tag to the analyte-bound nucleic acid forms a probe-nucleic acid complex; (c) amplifying the probe-nucleic acid complex to form an amplified probe-nucleic acid product; and (d) detecting the amplified probe-nucleic acid product. In some embodiments, the probe will contain a second oligo tag that can attach to an opposite end of the analyte bound nucleic acid complex. In some embodiments, the single oligo tag or the pair of oligo tags will comprise one or more unique identification sequences.

In some embodiments the methods comprise: (a) contacting a sample with a first probe comprising a first oligo tag and a first analyte binding domain and a second probe comprising a second oligo tag and second analyte binding domain, wherein said first and second probes can bind to a first and second analyte, respectively, such that said first and second oligo tags are in spatial proximity to form a complex with a first and second nucleic acid if said nucleic acid is bound by said first and second analyte in said sample; (b) attaching said first and second oligo tags to said first and second nucleic acid to form a probe-nucleic acid template; (c) amplifying said probe-nucleic acid template; and (d) detecting the presence or absence of an amplified probe-nucleic acid template.

Alternatively, methods for identifying an analyte-bound nucleic acid (e.g., nucleic acid bound by an analyte) in a sample may comprise: (a) contacting a sample with a first probe comprising a first and second oligo tag and a first analyte binding domain; and a second probe comprising a third and fourth oligo tag and a second analyte binding domain, wherein said second oligo tag provides a region of complementarity for said third oligo tag and one end of a nucleic acid and said fourth oligo tag provides a region of complementarity for said first oligo tag and another end of a nucleic acid, wherein said first and second probes can bind to an analyte, such that said first and second oligo tags and said third and fourth oligo tags are in spatial proximity to form a complex with said nucleic acid if said nucleic acid is bound by said analyte in said sample; (b) attaching said second and fourth oligo tags to said nucleic acid to form a probe-nucleic acid template; (c) amplifying said probe-nucleic acid template; and (d) detecting the presence or absence of an amplified probe-nucleic acid template.

In some embodiments, the second and fourth oligo tags provide a “bridge” or a “splint” between the ends of the nucleic acid and the first and fourth oligo tags. This permits localization of the ends of the first and third oligo tags adjacent to the respective ends of the nucleic acid for ligation. The numbering of the nucleic acids in the probes is interchangeable and is not intended to be limiting. Also, the location of the nucleic acids on the probes can be changed. Accordingly, in some instances, the first and third oligo tags may be located on said first probe and said second and fourth oligo tags may be located on said second probe. in one embodiment, one of the probes may comprise a first and second “bridge” oligo tag.

In another embodiment, the first probe may comprise a first oligo tag and a first analyte binding domain and a second probe comprising a second oligo tag and second analyte binding domain, wherein said first oligo tag provides a region of complementarity for said second oligo tag and an end of a nucleic acid.

In another embodiment, a single probe may be used. The probe may comprise an oligo tag and an analyte binding domain, wherein said oligo tag may be ligated to an end of a nucleic acid. The oligo tag may provide a region of complementarity to an end of a nucleic acid.

In another embodiment, after the proximity ligation step, nucleic acids in the mixture can be circularized by ligation under dilute conditions. After circularization, a target nucleic acid ligated to a probe nucleic acid may be specifically amplified by either rolling circle amplification using a single primer that is complementary to a sequence in the oligo tag or by PCR using two primers, where the first primer is complementary to a sequence on the Watson strand of the oligo tag and the second primer is complementary to a sequence on the Crick strand of the oligo tag. In this way, a target nucleic acid can be specifically amplified if either (or both) of the following conditions is met: (1) one end of the target nucleic acid has been ligated to a probe nucleic acid (oligo tag); (2) both ends of the target nucleic acid have each been ligated to a probe nucleic acid (oligo tag). In order to provide a free DNA end at the end of ligated oligo tags, one of the following may be done: (1) A restriction endonuclease cleavage site may be incorporated into the oligo tag so that the blocked end of the oligo tag that is tethered to the analyte binding domain can be removed by digesting the nucleic acids with the restriction endonuclease; (2) A modified nucleotide or base may be incorporated into the oligo tag so that the blocked end of the oligo tag that is tethered to the analyte binding domain can be removed by treating the nucleic acids with an appropriate enzyme or chemical that cleaves the oligo tag at the modified nucleotide or base to yield a 5′-phosphate, 5′-hydroxyl, or 3′-hydroxyl group; (3) The oligo tag may be tethered to the analyte binding domain by an internal linkage, e.g., via a modified base, leaving both ends of the oligo tag free for ligation or other treatments. The linker tethering the analyte binding domain to the oligo tag may include a moiety that can be cleaved with a chemical or enzyme to separate the analyte binding domain from the oligo tag, so that the oligo tag can serve as a template for DNA polymerase in an amplification reaction. In an alternative approach, the Watson and Crick strands of nucleic acids may be joined into a single linear template or may be circularized by ligating one or more linker oligonucleotides that form hair-pin structures in solution to one or both ends of the nucleic acids.

In various aspects, disclosed herein are kits and compositions for detecting analyte-bound nucleic acids. In some instances, the composition may be a probe comprising: (a) an analyte binding domain; and (b) an oligo tag. The oligo tag may comprise a linker compatible region, unique identification sequence, a primer binding site, or any combination thereof. The analyte binding domain may be an antibody.

Further disclosed herein is a kit comprising: a plurality of probes, wherein the probe comprises an analyte binding domain and an oligo tag. The oligo tag may comprise a linker compatible region, unique identification sequence, a primer binding site, or any combination thereof. The analyte binding domain may be an antibody. The kits disclosed herein may further comprise a plurality of linkers. Additionally, the kits may include a ligating agent. In other embodiments, the kits may comprise instructions for attaching the probe to an analyte-bound nucleic acid.

In one embodiment, the methods disclosed herein further comprise the step of cross-linking the analyte to the target nucleic acid in said sample with an agent that can cross-link said analyte to said target nucleic acid prior to contacting the analyte with said first and second probes. In one embodiment, the cross-linking agent is formaldehyde. Cross-linking may also comprise the sequential treatment of cells with a cross-linking agent, followed by treatment with formaldehyde. For example, cells may be treated with a bifunctional protein-protein cross-linking agent such as dimethyl adipimidate, dimethyl pimelimidate, ethylene glycol bis[succinimidylsuccinate], or disuccinimidyl glutarate followed by treatment with formaldehyde.

Generally, cross-linking may be about 2 to about 45 minutes, about 2 to about 30 minutes, about 5 to about 30 minutes, about 5 to about 20 minutes, about 5 to about 10 minutes, about 10 to about 30 minutes, about 10 to about 20 minutes. Additional agents may be added to terminate the cross-linking reaction. For example, glycine can be added to quench the formaldehyde and terminate the cross-linking reaction. It will be appreciated by those of skill in the art that other suitable cross-linking methods may also be used. Cross-linking the analyte to the target nucleic acid may occur by a variety of methods including, but not limited to, ultra-violet (UV) cross-linking, photochemical cross-linking and chemical cross-linking. Ultra-violet cross-linking may comprise irradiation of analyte-nucleic acid complexes with ultraviolet light thereby causing covalent bonds to form between the nucleic acid and analytes that are in close contact with the target nucleic acid. UV cross-linking may also include UV laser cross-linking. Photochemical crosslinking methods have long been used to identify and characterize protein-DNA interactions, and can be carried out by introducing a photo-reactive group into the DNA or the protein. Photochemical cross-linking may involve incorporation of a photoactivatable crosslinking agent at a single site within the target nucleic acid binding motif of the analyte (e.g., sequence-specific DNA binding protein), formation of the derivatized analyte-DNA complex, and UV-irradiation of the derivatized analyte-DNA complex. Solid phase synthesis or DNA polymerases may be used to incorporate a photo-reactive moiety into the target nucleic acid or the analyte. Alternatively, photocrosslinkers can be introduced into the analyte (e.g., DNA binding proteins) by chemical modification or semisynthesis, or by in vitro protein translation systems using chemically aminoacylated tRNAs. Chemical cross-linking may include the use of cross-linking agents. Suitable cross-linking agents include cisplatin, dimethyl adipimidate (DMA), dimethyl pimelimidate (DMP), dimethyl suberimidate (DMS), disuccinimidyl suberate (DSS), disuccinimidyl glutarate (DSG), ethylene glycol bis(succinimidylsuccinate) (EGS), Tris-succinimidyl aminotriacetate (TSAT), and formaldehyde. Additional cross-linking agents include alkylating agents (e.g., 1,3-bis(2-chloroethyl)-1-nitrosourea, nitrogen mustard), nitrous acid, malondialdehyde, psoralens, and aldehydes (e.g., acrolein, crotonaldehyde).

The target nucleic acid may be processed by a variety of methods, thereby forming a processed target nucleic acid-analyte complex. For example, the target nucleic acid-analyte complex may be extracted and/or purified from a cell. The target nucleic acid-analyte complex may undergo fragmentation. The processed target nucleic acid-analyte complex may undergo repair to the damaged target nucleic acid.

In another embodiment, the method further comprises the step of fragmenting the target nucleic acid prior to contacting the analyte with said first and second probes. In one embodiment, fragmentation is performed after said cross-linking step. Fragmentation may be performed before the cross-linking step. The target nucleic acid may be fragmented by sonication, needle shear, nebulization, acoustic/mechanical shearing, point-sink shearing, passage through a French pressure cell, or enzymatic digestion. Enzymatic digestion of the target nucleic acid or the cross-linked nucleic acid-analyte complex may occur by nuclease digestion, including but not limited to digestion with micrococcal nuclease, restriction endonucleases, exonucleases, RNase (e.g., RNAse H) or DNase (e.g., DNase I). In one embodiment the target nucleic acid is fragmented by sonication. Fragmentation of the target nucleic acid may result in fragment sized of about 100 bp to about 2000 bp, about 200 bp to about 1500 bp, about 200 bp to about 1000 bp, about 200 bp to about 500 bp, about 500 bp to about 1500 bp, and about 500 bp to about 1000 bp. In some embodiments, the target nucleic acid fragment size is about 200 bp to 500 bp.

The analyte-bound nucleic acid in the sample may be damaged in some cases and may be repaired by any suitable method known in the art. For example, after fragmentation or after cross-linking, the end of the target nucleic acid fragments may be damaged. In another example, the ends of the target nucleic acid may be repaired to facilitate ligation by any suitable method known in the art. Examples include, but are not limited to, generating ends that are compatible with ligation. The ends of the target nucleic acid may be repaired by an end-repair method. The target nucleic acid may be treated with one or more enzymes, for example, a nuclease.

In one embodiment, such an end-repair may comprise the formation of blunt ends at each end of the target nucleic acid fragments. Blunt ends may be achieved using methods known in the art, for example by using enzymes having exonuclease and polymerase activity, for example T4 DNA polymerase or Klenow DNA polymerase (either alone or together with T4 DNA polymerase) together with suitable dNTPS, for example, dATP, dTTP, dCTP and dGTP. In another example, the end-repair method may comprise the formation of sticky ends at one or both ends of the target nucleic acid or target nucleic acid fragments. In one embodiment, T4 polynucleotide kinase together with ATP may be included to ensure that free 5′-hydroxyl groups on the blunt ends are phosphorylated, suitable for ligation. In one example, once the target nucleic acid has been repaired, the T4 DNA polymerase, Klenow DNA polymerase, T4 polynucleotide kinase, or other enzymes used for the repair reactions can be inactivated by incubating at an elevated temperature for a fixed period of time (for instance, 75° C. for 20 min), generally following the recommendations of the enzyme manufacturers. Alternatively, the enzymes can be removed by column purification.

In one embodiment, the method further comprises the step of modifying said target nucleic acid to form 3′ or 5′ single stranded nucleic acid overhangs or linkers at the ends of the target nucleic acid.

Furthermore, the analyte-bound nucleic acid or any processed forms thereof may be modified by attaching an overhang or a linker to one or both ends of the target nucleic acid, thereby forming a linker-target nucleic acid complex. In another embodiment, the method further comprises the step of modifying said target nucleic acid by adding an overhang or linker to form a linker-target nucleic acid complex, wherein said linker-target nucleic acid complex comprises 3′ or 5′ single stranded nucleic acid overhangs at the ends of the nucleic acid.

Linkers or overhangs may comprise nucleic acids (e.g., RNA, DNA, RNA-DNA hybrids), peptide nucleic acids (PNAs), that comprise purine and pyrimidine bases, or other natural, chemically or biochemically modified, non-natural, or derivatized nucleotide bases. The nucleic acids may be single-stranded or double-stranded. The linker or overhang may be a single nucleotide (e.g., deoxyadenosine, deoxycytosine, deoxyguanosine, deoxythymidine). The linker may contain only one type of nucleotide (e.g., oligodT or oligodA). The linker or overhang may contain two or more different nucleotides (e.g., CTAG, CT, CA, TA, GC, GT, GA). The linker or overhang may be about 5 to about 50 nucleotides, about 5 to about 40 nucleotides, about 5 to about 30 nucleotides, about 5 to about 20 nucleotides, or about 5 to about 10 nucleotides. The linker or overhang may be attached to the target nucleic acid by ligation (e.g., blunt end ligation, sticky end ligation), hybridization, or PCR. One or more linkers or overhangs may be attached to the target nucleic acid. The linkers or overhangs may be attached to one or both ends of the target nucleic acid. In one example, the linkers or overhangs are non-complementary.

In one embodiment, the 3′ or 5′ single stranded nucleic acid overhangs are added to blunt ends of the target nucleic acid. The 3′ or 5′ single stranded nucleic acid overhangs may be added to the blunt ends by ligating a first linker to a first blunt end and a second linker to a second blunt end, wherein the first and second linkers are non-complementary. This prevents self-ligation of the target nucleic acid. Linkers may comprise nucleic acids (e.g., RNA, DNA, RNA-DNA hybrids), peptide nucleic acids (PNAs), that comprise purine and pyrimidine bases, or other natural, chemically or biochemically modified, non-natural, or derivatized nucleotide bases. The nucleic acids may be single-stranded or double-stranded. The linker may range in length from about 5 bp to about 50 bp or longer. In another example, the linker is attached to sticky ends of the target nucleic acid molecule. The linker may be attached by ligating a first linker to a first sticky end and a second linker to a second sticky end, wherein the first and second linkers are non-complementary. In another example, the linker may be attached to by ligating a first linker to a sticky end and a second linker to a blunt end, wherein the first and second linkers are non-complementary.

Alternatively, such overhangs or linkers may comprise a deoxyadenosine (dA). For example, an overhang or linker comprising a deoxyadenosine may be attached to a nucleic acid to form a linker-nucleic acid complex, wherein said linker-nucleic acid complex comprises a single base 3′ overhang of deoxyadenosine (3′-dA). In some cases, an enzyme, such as a DNA polymerase that lacks or has reduced exonuclease activity (e.g., exonuclease-defective mutant of Klenow DNA polymerase (Klenow exo-), Tag DNA polymerase, or Tth DNA polymerase) may be used to form the 3′ overhang of deoxyadenosine (3′-dA).

Hybridization may be used to attach linkers to target nucleic acids or to attach probes to linkers. Generally, the single-stranded portion of the linkers and target nucleic acids or the single-stranded portions of the probes and linkers may bind together at regions of sequence complementarity.

In one embodiment according to the first aspect, the first linker provides a first region of complementarity for said first nucleic acid or first oligo tag of said first probe and the second linker provides a second region of complementarity for said second nucleic acid or second oligo tag of said second probe. In one embodiment, the first linker and first nucleic acid form a first ligation complex at one end of the target nucleic acid and the second linker and second nucleic acid form a second ligation complex at another end of the target nucleic acid.

In another embodiment, in accordance with the second aspect, said second nucleic acid provides a region of complementarity for said third nucleic acid and a first linker at one end of said target nucleic acid and said fourth nucleic acid provides a region of complementarity for said first nucleic acid and a second linker at another end of said target nucleic acid. In one embodiment, said second nucleic acid, said third nucleic acid and said linker form a first ligation complex at one end of the target nucleic acid and said fourth nucleic acid, said first nucleic acid and said linker form a second ligation complex at another end of said target nucleic acid. In other words, the first linker and first oligo tag can form a first ligation complex at one end of the analyte-bound nucleic acid and the second linker and second oligo tag can form a second ligation complex at another end of the analyte-bound nucleic acid.

In another embodiment, said second oligo tag provides a region of complementarity for said third oligo tag and a first linker at one end of said analyte-bound nucleic acid and said fourth oligo tag provides a region of complementarity for said first oligo tag and a second linker at another end of said analyte-bound nucleic acid. In one embodiment, said second oligo tag, said third oligo tag and said linker form a first ligation complex at one end of the analyte-bound nucleic acid and said fourth oligo tag, said first oligo tag and said linker form a second ligation complex at another end of said analyte-bound nucleic acid.

In some instances, the oligo tag comprises a linker compatible region. The linker compatible region or a portion thereof may be able to hybridize to the linker on the target nucleic acid. The linker compatible region or a portion thereof may be complementary to the linker attached to the target nucleic acid. In one embodiment, the first linker provides a first region of complementarity for said first linker compatible region of said first probe and the second linker provides a second region of complementarity for said second linker compatible region of said second probe.

The target nucleic acid may comprise DNA, cDNA, single stranded DNA, double stranded DNA, plasmid DNA, RNA, mixtures of DNA with other molecules, DNA or RNA from human or other mammals. In one embodiment the nucleic acid is a single or double stranded nucleic acid. The nucleic acid may be RNA or DNA. In one embodiment, the target nucleic acid is DNA.

The oligo tag may further include a primer binding site. A primer may bind to the primer binding site and the oligo tag-target nucleic acid may be replicated by any of the methods disclosed herein. The primer binding site may hybridize to a universal primer. Examples of universal primers include, but are not limited to, T7 promoter, T7 terminator, T3, Sp6, M13F(−21), M13F(−40), M13R Reverse, AOX1 Forward, AOX1 Reverse, pGEX Forward (GST 5, pGEX 5′), pGEX Reverse (GST 3, pGEX 3′), BGH Reverse, GFP(C′ terminal, CFP, YFP, or BFP), GFP Reverse, GAG, GAG Reverse, CYC1 Reverse, pFastBacF, pFastBacR, pBAD Forward, pBAD Reverse, and CMV-Forward.

The analyte may be a protein or a nucleic acid or a mixture thereof. In some examples, the analyte may be a protein such as a transcription factor or a histone. Transcription factors may bind to the DNA and help initiate a program of increased or decreased gene transcription. Transcription factors include, but are not limited to, TFIIA, TFIIB, TFII2, TATA binding protein, TFIIE, TFIIF, or TFIIH. The analyte may contain DNA-binding domain, trans-activating domain, signal sensing domain, a zinc finger domain, helix-turn-helix motif, a leucine zipper and/or other domains that facilitate binding to a nucleic acid. The analyte may bind single-stranded or double-stranded nucleic acid molecules (e.g., ssDNA, dsDNA, ssRNA, dsRNA, DNA-RNA hybrid). For example, the analyte may be replication protein A. In another example, the analyte may be a histone acetyltransferase (HAT) protein or histone deacetylase (HDAC) protein. The analyte may comprise a plurality of plurality of proteins. For example, the analyte may comprise a transcription complex. The analyte may be an epitope shared by one or more proteins, a protein post-translational modification, an amino acid or peptide sequence shared by one or more proteins that includes zero, one, or more residues modified by a post-translational modification, a covalent modification to a protein or nucleic acid, a small molecule bound to a protein or nucleic acid, or an amino acid or nucleic acid analog incorporated into a protein or nucleic acid. The analyte may be a nucleic acid. The analyte may be any molecule bound or covalently linked to DNA, any molecule bound or covalently linked to DNA-associated proteins, or any molecule bound or covalently linked to other directly or indirectly DNA-associated molecules. The analyte may be any mixture of the foregoing examples. Residues in proteins modified by post-translational modifications include, but are not limited to, phosphoserine, phosphothreonine, phosphotyrosine, monomethyl arginine, symmetric dimethyl arginine, asymmetric dimethyl arginine, and mono-, di-, or trimethyl lysine. Examples of other protein post-translational modifications include, but are not limited to, acetylation, ubiquitination, SUMOylation, and disulfide bond formation. Analysis of the genome-wide distribution of histone-associated post-translational modifications is an important application of the method and is used to study epigenetic regulation of gene expression during development, cell lineage differentiation, and in the progression of diseases such as cancer. An example of a covalent modification of nucleic acids is cytosine methylation. Analysis of the genome-wide distribution of histone-associated post-translational modifications and of cytosine methylation at CpG sites are important applications of the method that are used to study epigenetic regulation of gene expression during development, cell lineage differentiation, and in the progression of diseases such as cancer.

The analyte-bound nucleic acid (e.g., target nucleic acid, nucleic acid, nucleic acid bound by an analyte) may comprise DNA, cDNA, single stranded DNA, double stranded DNA, plasmid DNA, RNA, mixtures of DNA with other molecules, DNA or RNA from human or other mammals. In one embodiment the nucleic acid is a single or double stranded nucleic acid. The nucleic acid may be RNA or DNA. In one embodiment, the analyte-bound nucleic acid is DNA. In some instances, the analyte-bound nucleic acid is a nucleic acid bound by a histone protein. Alternatively, the analyte-bound nucleic acid is a nucleic acid bound by a transcription factor. The analyte-bound nucleic acid may also be a methylated nucleic acid. The analyte-bound nucleic acid may also contain one or more base analogs. Examples of base analogs include BrdU and BrdI, which may be incorporated by pulse-labeling cells and may serve as a marker of newly replicated DNA. The analyte-bound nucleic acid may contain covalent modifications, including modifications resulting from DNA damage. The analyte-bound nucleic acids or the sequences of the analyte-bound nucleic acids attached to the oligo tags of two or more probes may be essentially the same. For example, a nucleic acid may have multiple copies and a first end of a first copy of the nucleic acid may be attached to a first oligo tag of a first probe and a second end of the first copy of the nucleic acid may be attached to a second oligo tag of a second probe. The sequence of the analyte-bound nucleic acids may be at least about 20%, about 30%, about 40%, about 50%, about 60%, about 70%, about 80%, about 90%, about 95%, about 97%, about 100% identical. For example, the analyte-bound nucleic acid may be fragmented to produce fragments of different sizes and sequences of copies of an analyte-bound nucleic acid and the oligo tags of two or more different probes may attach to analyte-bound nucleic acids of similar sequences and/or sizes. In some instances, the sequences of the analyte-bound nucleic acids may be essentially the same; however, the analyte-bound nucleic acid molecules attached to the oligo tags of two or more probes may be different. For example, a nucleic acid may have multiple copies and a first copy of the nucleic acid may be attached to an oligo tag of a first probe, whereas a second copy of the nucleic acid may be attached to an oligo tag of a second probe. Alternatively, the sequences of the analyte-bound nucleic acids attached to the oligo tags of two or more probes may be different. For example, the attachment of a linker to the analyte-bound nucleic acid may alter the sequence of the analyte-bound nucleic acid. In another example, a first oligo tag of a first probe may attach to a first analyte-bound nucleic acid and a second oligo tag of a second probe may attach to a second analyte-bound nucleic acid, wherein the sequences of the first analyte-bound nucleic acid and second analyte-bound nucleic acid are different.

The analyte binding domain may comprise an aptamer, or antigen binding protein. In one embodiment, the aptamer is an oligonucleotide or a peptide aptamer.

In one embodiment the antigen binding protein is selected from the group consisting of antibodies, antibody fragments and other protein constructs, such as domains, which can bind to an antigen. In one embodiment, the antigen binding protein is an antibody.

In one embodiment the antibody is selected from the group consisting of monoclonal, recombinant, polyclonal, chimeric, humanized, bispecific and heteroconjugate antibodies; a single variable domain, a domain antibody, antigen binding fragments, immunologically effective fragments, single chain Fv, diabodies and TANDABS™.

The analyte binding domains of two or more probes may be at least about 50%, at least about 60%, at least about 70%, at least about 80%, or at least about 90% identical. The analyte binding domains of two or more probes may the same. The analyte binding domains of two or more probes may bind to the same analyte. For example, the analyte binding domains of two or more probes may bind to the same transcription factor. In another example, the analyte binding domains of two or more probes may bind to the same analyte and the analyte may bind to the same nucleic acids. Alternatively, the analyte binding domains of two or more probes may bind to the same analyte, however, the analyte may bind to two or more different nucleic acids. For example, the analyte binding domain of a first probe may bind to an SP1 transcription factor that is bound to a first nucleic acid and the analyte binding domain of a second probe may also bind to an SP1 transcription factor that is bound to a second nucleic acid, however, the first and second nucleic acids bound by the SP1 transcription factor may be different. The analyte may be at least about 20%, about 30%, about 40%, about 50%, about 60%, about 70%, about 80%, about 90%, about 95%, about 97%, about 100% identical. The analyte binding domains of two or more probes may bind to two or more different analytes. For example, the analyte binding domain of a first probe may bind to a transcription factor and the analyte binding domain of a second probe may bind to a transcriptional coactivator. The analyte binding domains may bind to an analyte that is directly bound to a nucleic acid. For example, the analyte binding domain may bind to a transcription factor that is directly bound to a nucleic acid. Alternatively, the analyte binding domains may bind to an analyte that is indirectly bound to a nucleic acid. For example, a transcription factor that is directly bound to a nucleic acid may recruit a transcriptional coactivator. The transcription coactivator may bind to the transcription factor, thereby indirectly binding to the nucleic acid. The analyte binding domain of the probe may bind to the transcriptional coactivator.

In one embodiment, the first analyte binding domain and said second analyte binding domain can specifically bind to the same analyte. In another embodiment, the first analyte binding domain and said second analyte binding domain can specifically bind to different analytes. In another embodiment, the first and second probes bind to the analyte simultaneously. In another embodiment, the first and second probes bind to the analyte sequentially.

In one embodiment, the ligation step may comprise a chemical ligation reaction or an enzymatic ligation reaction. In one embodiment, the ligation step is an enzymatic ligation reaction.

In one embodiment, the enzymatic ligation reaction comprises a ligase. In one embodiment, the ligase may be a template-dependent or template-independent ligase. In one embodiment according to the second aspect, the ligase is a template-dependent ligase. The ligase may be a protein ligase such as T4 DNA ligase, Escherichia coli DNA ligase, or Taq DNA ligase. Alternatively, the ligase may be a nucleic acid ligase, such as a ribozyme or deoxyribozyme. In one embodiment, the ligase is T4 DNA ligase. In some instances, the ligase is an RNA ligase such as T4 RNA ligase.

Methods of ligation are known to those of skill in the art and are described, for example in Sambrook et al. (2001) and the New England BioLabs catalog both of which are incorporated herein by reference for all purposes. Methods include using T4 DNA ligase which catalyzes the formation of a phosphodiester bond between juxtaposed 5′ phosphate and 3′ hydroxyl termini in duplex DNA or RNA with blunt and sticky ends; Taq DNA ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′→5′ phosphodiester bond, substrates include single-stranded RNA and DNA as well as dinucleoside pyrophosphates; or any other methods described in the art.

The nucleic acids located on the probes as described herein may contain unique nucleic acid sequences suitable for amplification of the ligated target nucleic acid template by isothermal amplification or PCR using nucleic acid primers that specifically recognize the unique nucleic acid sequences in the nucleic acids located on the probes. Alternatively, the primers may recognise the unique nucleic acid sequences in the nucleic acids located on the probes together with a sequence located within the target nucleic acid (locus specific amplification).

The target nucleic acid may be amplified by polymerase chain reaction (PCR) or isothermal amplification. An example includes, Mattila et al., Nucleic Acids Res. 19, 4967 (1991) which is incorporated herein by reference in its entirety. PCR-based amplification methods may comprise PCR, real-time PCR, reverse-transcription PCR, quantitative PCR, emulsion PCR, droplet PCR, hot start PCR, in situ PCR, inverse PCR, multiplex PCR, Variable Number of Tandem Repeats (VNTR) PCR, asymmetric PCR, long PCR, nested PCR, hemi-nested PCR, touchdown PCR, assembly PCR, colony PCR, or digital PCR. Amplification methods can include PCR. Non-PCR-based amplification methods may also be utilized and may comprise multiple displacement amplification (MDA), transcription-mediated amplification (TMA), nucleic acid sequence-based amplification (NASBA), strand displacement amplification (SDA), real-time SDA, rolling circle amplification, or circle-to-circle amplification.

In one embodiment, the ligated nucleic acid template may be denatured prior to amplification. In one embodiment, the denaturation comprises heat treatment. The heat treatment is carried out at a temperature in the range selected from the group consisting of from about 70-95° C.; about 75-95° C.; about 80-95° C. and about 85-95° C. In one embodiment, the denaturation step is carried out at 95° C.

In one embodiment, the denaturation step is carried out for a period selected from the group consisting of from about 1-30 minutes; about 1-20 minutes and about 1-10 minutes. In one embodiment, the denaturation step is carried out for at least 3 minutes. The amplification step may comprise a polymerase chain reaction or isothermal amplification.

In one embodiment, the amplifying step comprises a polymerase chain reaction (PCR). The PCR may comprise 15 cycles at 95° C. for 30 seconds, 65° C. for 30 seconds, 72° C. for 2 min, and a final extension at 72° C. for 10 min.

Detection and quantification of the relative amounts of the amplified nucleic acid template may be by quantitative PCR (qPCR), including real-time quantitative PCR. In one embodiment, the amplified target nucleic acid template is detected with a nucleic acid dye suitable for real-time qPCR such as SYBR Green or EvaGreen. In an alternative embodiment, the amplified target nucleic acid template is detected with nucleic acid dye such as ethidium bromide.

In an alternative embodiment, the primers used in the qPCR detection may comprise a detectable label. Examples of labels that may be used include, but are not limited to: fluorescent markers or reporter dyes, for example, 6-carboxyfluorescein (6FAM™), Maxima Sybergreen (Fermentas), NED™ (Applera Corporation), HEX™ or VIC™ (Applied Biosystems); TAMRA™ markers (Applied Biosystems, CA, USA); chemiluminescent markers, for example Ruthenium probes; and radioactive labels, for example tritium in the form of tritiated thymidine. ³²-Phosphorus or ³³-Phosphorus may also be used as a radiolabel.

Alternatively the detectable label may be directly or indirectly attached to the primer and may be selected from the group consisting of electroluminescent tags, magnetic tags, affinity or binding tags (such as biotin, avidin, streptavidin, HRP, protein A, protein G, antibodies or fragments thereof, polyhistidine, Ni²⁺, FLAG tags, myc tags), nucleotide sequence tags, position specific tags, fluorescent tags, chemiluminescent tags, fluorescent quenching agents, gold or heavy metal tags, enzymes (examples include alkaline phosphatase, peroxidase and luciferase), electron donors/acceptors, acridinium esters, dyes, calorimetric substrates, scintillants, and/or tags with specific physical properties such as different size, mass, gyration, ionic strength, dielectric properties, polarisation or impedance.

The methods as described herein are suitable for use, in some embodiments, in a sample of fresh tissue, frozen tissue, paraffin-preserved tissue and/or ethanol preserved tissue. The sample may be a biological sample. Non-limiting examples of biological samples include whole blood or a component thereof (e.g. plasma, serum), urine, saliva lymph, bile fluid, sputum, tears, cerebrospinal fluid, bronchioalveolar lavage fluid, synovial fluid, semen, ascitic tumor fluid, breast milk, pus and chromatin. In one embodiment the target nucleic acid sample is chromatin.

The amplified target nucleic acids may be detected and the relative abundance of specific target nucleic acids may be estimated by sequencing methods such as high-throughput Next Generation DNA sequencing. Examples of high-throughput sequencing include, but are not limited to, Lynx Therapeutics' Massively Parallel Signature Sequencing (MPSS), Polony sequencing, 454 pyrosequencing, Illumina (Solexa) sequencing, SOLiD sequencing, Ion semiconductor sequencing (Ion Torrent), DNA nanoball sequencing, Helioscope™ single molecule sequencing, Single Molecule SMRT™ sequencing, Single Molecule real time (RNAP) sequencing, Nanopore DNA sequencing, or VisiGen Biotechnologies sequencing.

The nucleic acids located either on the probes as described herein or on the primers used to specifically amplify the target nucleic acids ligated to the probes as described herein may contain unique nucleic acid sequences suitable for processing the ligated target nucleic acid for high-throughput Next Generation DNA sequencing or library preparation for Next Generation DNA sequencing.

The nucleic acids located on the probes as described herein may contain a randomized or partially randomized sequence that can be used as a nucleotide “bar code” or unique sequence identifier (e.g., unique identification sequence). Alternatively, a nucleotide bar code or unique sequence identifier can be added after the ligation step by PCR using primers complementary to the probe nucleic acid and a limited number of PCR cycles (about 2 PCR cycles). Tagging each of the probe nucleic acid-target nucleic acid ligation products with a unique sequence identifier may enable the absolute quantitation of DNA fragments and/or analytes or improve the accuracy or sensitivity with which the relative abundance of the DNA fragments and/or analytes may be determined based on high-throughput sequencing data.

The unique sequence identifier (e.g., unique identification sequence) may be attached to the target, for example, by a stochastic process. The unique identification sequence may be attached to the target by a non-random process. Generally, the unique identification sequence is attached to the target prior to amplification of the target nucleic acid. When the target is the target nucleic acid or analyte, the unique identification sequences may be attached to the target after fragmentation of the target nucleic acid, after attachment of the linker, after attachment of the probe, or prior to amplification of the target nucleic acid. When the target is the linker, probe, analyte binding region, or oligo tag, the unique identification sequences may be attached to the target in any step prior to amplification of the target nucleic acid. For example, the unique identification sequence may be incorporated directly into the linker or probe prior to attachment of the linker or the probe to the target nucleic acid or the linker, respectively.

The unique identification sequence may comprise nucleic acids (e.g., RNA, DNA, RNA-DNA hybrids), peptide nucleic acids (PNAs), that comprise purine and pyrimidine bases, or other natural, chemically or biochemically modified, non-natural, or derivatized nucleotide bases. The nucleic acids may be single-stranded or double-stranded. The unique identification sequence may be a single nucleotide (e.g., deoxyadenosine, deoxycytosine, deoxyguanosine, deoxythymidine). The unique identification sequence may contain only one type of nucleotide (e.g., oligodT or oligodA). The unique identification sequence may contain two or more different nucleotides (e.g., CTAG, CT, CA, TA, GC, GT, GA). The unique identification sequence may be about 5 to about 50 nucleotides, about 5 to about 40 nucleotides, about 5 to about 30 nucleotides, about 5 to about 20 nucleotides, or about 5 to about 10 nucleotides.

The unique identification sequence may be incorporated into the probe. For example, the probe may comprise an analyte binding region, oligo tag, and unique identification sequence. In another example, the probe comprises an analyte binding region and oligo tag, wherein the oligo tag comprises a linker compatible region and a unique identification sequence. Probes specific for different analytes may be distinguished by the presence of the unique identification sequence. For example, the unique identification sequence for a first probe specific for a first analyte may be different for a second probe specific for a second analyte, wherein the first and second analytes are different. Probes specific for the same analyte, but different analyte binding regions, may also be distinguished by the presence of the unique identification sequence. For example, the unique identification sequence for a first probe comprising a first analyte binding region may be different for a second probe comprising a second analyte binding region, wherein the first and second analyte binding regions are different.

Probes specific for the same analyte binding region may be distinguished by the presence of the unique identification sequence. For example, the unique identification sequence for a first probe comprising a first analyte binding region may be different for a second probe comprising a second analyte binding region, wherein the first and second analyte binding regions are identical or similar. The unique identification sequence may be a nucleic acid sequence which may distinguish different target nucleic acids and/or identical or similar target nucleic acids. For example, the unique identification sequence for a first target nucleic acid may be different from the unique identification sequence for a second target nucleic acid molecule, wherein the sequences of the first and second target nucleic acids are different. Thus, the unique identification sequence can be used to distinguish different target nucleic acid molecules.

In another example, the unique identification sequence for a first target nucleic acid may be different from the unique identification sequence for a second target nucleic acid molecule, wherein the sequences of the first and second target nucleic acids are identical or similar. Thus, the unique identification sequence can be used to distinguish individual occurrences of a target nucleic acid. The unique identification sequence may also be used to distinguish different analytes. For example, the unique identification sequence for a first analyte may be different from the unique identification sequence for a second analyte, wherein the first and second analyte are different.

The unique identification sequence may also be used to distinguish individual occurrences of an analyte. For example, the unique identification sequence for a first analyte may be different from the unique identification sequence for a second analyte, wherein the first and second analyte are identical or similar. The unique identification may comprise a random sequence, predetermined sequence, degenerate sequence, semi-degenerate sequence, mixed sequence, ambiguous sequence, or wobble sequence. In some embodiments, the unique identification comprises a random sequence.

In some embodiments an amplified unique identification can be hybridized to an array. Signal from the array can then, for example, be used to count unique occurrences of a bound analyte.

In some embodiments the methods provided here are used to detect and/or quantify many analyte-bound nucleic acids simultaneously. For example a pool of probes label with barcodes can be employed to bind to, amplify, and detect multiple analyte-bound nucleic acids simultaneously. In some embodiments the associated binding and amplification can take place in a single reaction vessel. High-throughput next generation sequencing can then be used to identify analyte-bound nucleic acids which may be associated with a particular bar code. In some embodiments stochastically added unique identification's can be used to label the pool of probes allowing the digital counting of multiple analyte-bound nucleic acids or analyte binding events. In some embodiments arrays are used to detect the results of multiplexed probe binding.

The methods disclosed herein may further comprise separating, removing, deactivating, or denaturing proteins and/or analytes from the sample. In some cases, the method may further comprise terminating or reversing the cross-linking reaction, thereby uncross-linking the analyte and nucleic acid. For example, the cross-linked sample may be placed incubated in a high salt solution (e.g., 5M sodium chloride (NaCl)) at about 65° C. for at least about 2 hours (e.g., at least about 3 hours, 4 hours, 5 hours, or 6 hours), thereby reversing the cross-linked reaction. In another example, samples may be heated at about 90° C. to about 100° C. for a period of time sufficient to reverse the cross-linking between the analyte and the nucleic acid. The samples may be heated for at least about 5 min, 10 min, 20 min, 30 min, 40 min, 50 min, or 60 min.

The methods described herein may be suitable for use, in some embodiments, in a sample of fresh tissue, frozen tissue, paraffin-preserved tissue and/or ethanol preserved tissue. The sample may be a biological sample. Biological samples may be of any biological tissue or fluid or cells from any organism. Frequently the sample will be a “clinical sample” which is a sample derived from a patient. Clinical samples provide a rich source of information regarding the various states of gene expression and copy number. Non-limiting examples of biological samples include whole blood or a component thereof (e.g. plasma, serum), urine, saliva lymph, bile fluid, sputum, tears, cerebrospinal fluid, bronchioalveolar lavage fluid, synovial fluid, semen, ascitic tumor fluid, breast milk, pus and chromatin. Biological samples may also include sections of tissues, such as frozen sections or formalin-fixed sections taken for histological purposes, which may include formalin-fixed, paraffin embedded (FFPE) samples and samples derived therefrom. FFPE samples are a particularly important source for study of archived tissue as they can nucleic acids can be recovered from these samples even after long term storage of the samples at room temperature. See, for example, Specht et al. Am J. Path. (2001), 158(2):419-429, incorporated herein by reference. The sample may be from a tissue or organ such as a kidney, liver, pancreas, adrenal gland, brain, bladder, gallbladder, esophagus, heart, ear, skin, breast, lung, muscle, nose, mouth, eye, trachea, stomach, uterus, thyroid gland, spleen, thymus, small intestine, stomach, pineal gland, bone, tendon, or vermiform appendix. The sample may also be from a mammal (e.g., human, monkey, ape, pig, dog, cat, rodent, horse, cow, sheep, goat, rabbit), or non-mammal (e.g., amphibian, newt, lizard, snake, or reptile).

A biological sample as contemplated herein includes cultured biological materials, including a sample derived from cultured cells, such as a cell pellet. Accordingly, a biological sample may refer to a lysate, homogenate or extract prepared from a whole organism or a subset of its tissues, cells, isolated DNA-containing organelles such as nuclei or mitochondria, or component parts, or a fraction or portion thereof. A biological sample may also be modified prior to use, for example, by purification of one or more components, dilution, and/or centrifugation.

Well-known extraction and purification procedures are available for the isolation of nucleic acid from a sample. The nucleic acid may be used directly following extraction from the sample.

The methods as described herein may be suitable in some embodiments for detecting a wide variety of analytes such as protein covalently linked to DNA by in vivo crosslinking; post-translational modifications of proteins that are covalently linked to DNA by in vivo cross linking; covalent modifications of DNA that regulate or correlate with patterns of gene expression or that result from DNA damage, for example, CpG methylation, 7,8-dihydro-8-oxoguanine (8-oxoG) or pyrimidine (6-4) pyrimidone photoproducts and cyclobutane pyrimidine dimmers (CPD); synthetic bases incorporated into DNA such as bromodeoxyuridine (BrdU) and iododeoxyuridine (BrdI), which are used to label newly replicated DNA.

In one embodiment, the method as described herein is suitable to identify and quantitate the abundance of DNA fragments that are covalently linked to two different analytes using antibodies that can bind to each analyte, wherein each antibody is tethered to a different oligonucleotide sequence. Use of probes comprising a unique identification may enable the absolute quantitation of DNA fragments and/or analytes. The relative abundance of the DNA fragments and/or analytes may also be determined. In accordance with this embodiment the method may be used to identify DNA fragments bound by two specific proteins; or to identify DNA fragments bound by a protein that is in complex with a second specific protein; or to identify DNA fragments bound or associated with a protein that carries a specific covalent modification using an antibody specific for the protein and an antibody specific for the covalent modification, for example, identifying sites bound by an unidentified protein (X) phosphorylated on a tyrosine using anti-protein X and anti-phosphotyrosine antibodies. In some embodiments, it is possible to use antibodies that can bind the same post-translational modification on many different proteins rather than having to generate or use antibodies that only recognize the post-translational modification in the context of a specific protein sequence (e.g., a tyrosine phosphorylation consensus site).

In another embodiment, the method as described herein is suitable for the identification of newly replicated DNA fragments bound to a specific analyte, wherein the newly replicated DNA may be pulse labeled with BrdU and the corresponding DNA fragments are specifically tagged using an oligonucleotide-tethered anti BrdU antibodies.

In another embodiment, the method as described herein is suitable for the identification of DNA fragments involved in methylation complexes, wherein one of the antibodies may recognize methylated DNA and another may recognize a protein bound adjacent or near to the methylated DNA.

In another embodiment, the method described herein is suitable for improving the affinity, avidity, or specificity of antibodies or aptamers. For example, the affinity for a first probe comprising a first analyte binding domain and a first oligo tag may be improved with the addition of a second probe comprising a second analyte binding domain and a second oligo tag. In some cases, the affinity, avidity, or specificity of the analyte binding domain may be improved by at least about 20%, about 30%, about 40%, about 50%, about 60%, about 70%, about 80%, about 90%, or about 95%. In some instances, probes may comprise two pools of analyte binding domains (e.g., antibody or aptamer) are conjugated to two different oligo tags. Alternatively, probes may comprise two different analyte binding domains (e.g., antibodies or aptamers) that recognize the same analyte (e.g., antigen) are each conjugated to a different oligonucleotide probe.

In another embodiment, the sensitivity of the method may be increased by using competitor DNAs that can ligate to the chromatin ends and block ligation of antibody-conjugated oligonucleotides. By titrating the concentration of the competitor DNAs, a concentration may be found that suppresses non-specific proximity-independent ligation.

In some embodiments, any modified DNA that can be recognized by an antibody can be interrogated as to which proteins are binding near to the modified DNA.

In some embodiments, the steps of the method as described herein can be performed in a single EPPENDORF® tube without any physical separation steps. The method as described herein permits a high-throughput alternative to conventional chromatin immunoprecipitation that can be readily automated.

In some embodiments, the method as described herein can be applied to single cells for performing ultrasensitive epigenetic analysis.

In some embodiments, the method as described herein may be used for identifying genomic loci in a sample. It may also be used to identify what specific loci are bound by a particular protein.

Also disclosed herein are compositions and kits for identifying analyte-bound nucleic acids in a sample comprising: (a) a first container comprising a first probe, wherein said first probe comprises a first oligo tag and a first analyte binding domain; (b) a second container comprising a second probe, wherein said second probe comprises a second oligo tag and second analyte binding domain; and (c) instructions for using the first and second probes to detect an analyte in a sample.

Further disclosed herein are compositions and kits for identifying analyte-bound nucleic acids in a sample comprising: (a) a first container comprising a first probe comprising a first and second oligo tag and a first analyte binding domain; (b) a second container comprising a second probe comprising a third and fourth oligo tag and a second analyte binding domain; and (c) instructions for using the first and second probes to detect an analyte in a sample.

In one embodiment, the kits further comprise a third container comprising a ligating agent.

In another embodiment, the first and third oligo tags or first and third nucleic acids are located on said first probe and said second and fourth oligo tags or second and fourth nucleic acids are located on said second probe.

In some instances, the second and fourth oligo tags or second and fourth nucleic acids provide a “bridge” or a “splint” between the ends of the target nucleic acid and the first and fourth oligo tags or first and fourth nucleic acids. This permits localization of the ends of the first and third nucleic acids adjacent to the respective ends of the target nucleic acid for ligation. It will be appreciated that the numbering of the nucleic acids in the probes is interchangeable and is not intended to be limiting. It will also be appreciated that the location of the nucleic acids on the probes can be changed. Accordingly, in one embodiment of the fourth aspect, the first and third nucleic acids may be located on said first probe and said second and fourth oligo tags or second and fourth nucleic acids may be located on said second probe.

Methods for treating a disease or condition characterized by aberrant gene expression or aberrant activity of a gene product is also disclosed herein. Generally, said methods comprise: (a) obtaining a sample from a subject in need thereof; (b) contacting the sample with a probe, wherein said probe comprises an analyte binding domain and an oligo tag; (c) attaching said oligo tag to an analyte-bound nucleic acid in said sample; (d) detecting the presence or absence of said analyte-bound nucleic acid; and (e) determining a treatment regimen based on the presence or absence of said analyte-bound nucleic acid. The method may further comprise administering, adjusting, or terminating a treatment.

In some instances, the disease or condition is a cancer. The cancer may be a malignant cancer. The cancer can be a leukemia or lymphoma. Leukemia includes, but are not limited to, acute lymphocytic leukemia, acute myelocytic leukemia, chronic lymphocytic leukemia, chronic myelocytic leukemia. Additional types of leukemias include hairy cell leukemia, chronic myelomonocytic leukemia, and juvenile myelomonocytic-leukemia. The lymphoma may be a Hodgkin lymphoma, previously known as Hodgkin's disease, and non-Hodgkin lymphoma. Examples of non-Hodgkin lymphomas include Burkitt's lymphoma and mycosis fungoides. Non-Hodgkin lymphomas are all lymphomas which are not Hodgkin's lymphoma. Non-Hodgkin lymphomas can be further divided into indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

Alternatively, the cancer may be a solid tumor, such as a sarcoma or carcinoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma). In some embodiments, the cancer is a schwannoma. In some embodiments, the schwannoma is a spontaneous schwannoma. In some embodiments, the schwannoma is a malignant scwhannoma. In some embodiments, the schwannoma is a bilateral vestibular scwhannoma. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penis cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. The cancer may be a skin cancer, such as a basal cell carcinoma, squamous cell carcinoma, nonmelanoma skin cancer, actinic (solar) keratosis. An actinic keratosis is a precancerous condition that can develop into squamous cell carcinoma, or melanoma. Alternatively, the cancer may be a CNS tumor such as a glioma or nonglioma. The glioma may be a malignant glioma, high grade glioma, or diffuse intrinsic pontine glioma. Nongliomas include meningiomas, pituitary adenomas, primary CNS lymphomas, and medulloblastomas. Additional examples of gliomas include astrocytomas, oligodendrogliomas (or mixtures of oligodendroglioma and astocytoma elements), and ependymomas. The cancer may be a recurrent cancer or a refractory cancer.

In one embodiment, provided herein is a probe, which can comprise:
an analyte binding domain; and
an oligo tag, wherein the oligo tag can comprise a linker compatible region.

In another embodiment, provided herein is a probe, which can comprise:
an analyte binding domain; and
an oligo tag, wherein the oligo tag can comprise a primer binding site.

In one embodiment, the analyte binding domain is an antibody. In another embodiment, the oligo tag can further comprise a unique identification sequence. The unique identification sequence can also be attached to the oligo tag by a stochastic method.

In another embodiment, provided herein is a pool of probes, wherein each probe can comprise:
an analyte binding domain; and
an oligo tag, wherein the oligo tag can comprise a linker compatible region.

In still another embodiment, provided herein is a pool of probes, wherein each probe can comprise:
an analyte binding domain; and
an oligo tag, wherein the oligo tag can comprise a primer binding site.

In another embodiment, the analyte binding domain can be an antibody. The oligo tag can further comprise a unique identification sequence. In one embodiment, the unique identification sequence can be attached to the oligo tag by a stochastic method. In a further example, the analyte binding domain of the probes can bind to the same analyte. In still another embodiment, the analyte binding domain of the probes can bind to two or more different analytes.

In a further embodiment, provided herein is an amplified polynucleotide, wherein the amplified polynucleotide can comprise:
an amplified product of a genomic region associated with an analyte;
unique identification sequence or an amplified product thereof, wherein the unique identification sequence can be associated with the identity of the analyte; and
an amplified product of a linker compatible region.

In one embodiment, the unique identification sequence can be associated with the identity of the analyte after the polynucleotide is amplified.

In another embodiment, provided herein is an amplified polynucleotide comprising:
an amplified product of a genomic region associated with an analyte, wherein the analyte can be bound by the probe as described herein;

In still a further embodiment, provided herein is a pool of amplified polynucleotides, wherein each amplified polynucleotide can comprise:
an amplified product of a genomic region associated with an analyte;
unique identification sequence or an amplified product thereof, wherein the unique identification sequence can be associated with the identity of the analyte; and
an amplified product of a linker compatible region.

In still another embodiment, provided herein is a kit comprising:
a probe comprising an analyte binding domain; and
a linker, wherein the linker can attach to an analyte-bound nucleic acid.

In another embodiment, provided herein is a kit, wherein the kit can comprise:
a probe comprising an oligo tag, wherein the oligo tag comprises a linker compatible region; and
a linker, wherein the linker is can attach to an analyte-bound nucleic acid.

The kit can further comprise a unique identification sequence. In another embodiment, the kit described herein, can further comprise a ligation agent. The kit can further comprise a unique identification sequence. In one embodiment, the probe can further comprise an oligo tag and the oligo tag can comprise a linker compatible region. In another embodiment, the probe can further comprise an analyte binding domain.

In still another embodiment, provided herein is a method, wherein the method comprises:
contacting a sample comprising a protein and a nucleic acid molecule with a first probe comprising a first analyte binding domain and a first oligo tag, wherein the first oligo tag comprises a linker compatible region;
attaching the first oligo tag to a first target nucleic acid, wherein attachment of the oligo tag to the target nucleic acid forms a probe-nucleic acid complex;
amplifying the probe-nucleic acid complex to form an amplified probe-nucleic acid product; and
detecting the amplified probe-nucleic acid product.

In still another embodiment, provided herein is a method, wherein the method comprises:
contacting a sample comprising a protein and a nucleic acid molecule with a first probe comprising a first analyte binding domain and a first oligo tag, wherein the first oligo tag comprises a unique identification sequence;
attaching the first oligo tag to a first target nucleic acid, wherein attachment of the oligo tag to the target nucleic acid forms a probe-nucleic acid complex;
amplifying the probe-nucleic acid complex to form an amplified probe-nucleic acid product; and
detecting the amplified probe-nucleic acid product.

In one embodiment, the oligo tag can comprise a linker compatible region. In another embodiment, the oligo tag can comprise a unique identification sequence. In another embodiment, the analyte binding domain is an antibody. In one embodiment, the attaching step can comprise ligating the first oligo tag and/or second oligo tag to the target nucleic acid.

The method can also comprise attaching a linker to the target nucleic acid. In one embodiment, the attaching step can comprise ligating the first oligo tag and/or second oligo tag to the linker. In another embodiment, the probe is directly attached to the analyte while in another embodiment, the probe is indirectly attached to the analyte.

While the making and using of various embodiments of the disclosure are discussed in detail below, it should be appreciated that the disclosure provides many applicable inventive concepts that can be embodied in a wide variety of specific contexts. The specific embodiments discussed herein are merely illustrative of specific ways to make and use the invention and do not delimit the scope of the invention.

Terms such as “a”, “an” and “the” may not be intended to refer to only a singular entity, but include the general class of which a specific example may be used for illustration.

The term “polymerase chain reaction (PCR)” may refer to an enzyme-mediated reaction use to amplify a specific target DNA sequence. By amplifying the target DNA sequence in the DNA template, it is then able to produce millions more copies of the targeted DNA sequence. This is useful when a biological sample contains only small amounts of DNA. PCR is carried out in a mixture containing DNA polymerase, a pair of primers (forward and reverse) and four deoxynucleotide triphosphates (dNTPs) with the aid of thermal cycler.

The term “complementary” or “complementarity”, as used herein, may refer to a nucleic acid sequence that is complementary to a specified nucleic acid sequence.

The term “primer”, as used herein, may refer to any single-stranded oligonucleotide sequence that can be used as a primer in, for example, PCR technology. Thus, a “primer” according to the disclosure refers to a single-stranded oligonucleotide sequence that can act as a point of initiation for synthesis of a primer extension product that is substantially identical to the nucleic acid strand to be copied (for a forward primer) or substantially the reverse complement of the nucleic acid strand to be copied (for a reverse primer). A primer may be suitable for use in, for example, PCR technology.

As used herein, the term “isolated” may refer to a nucleic acid molecule, gene, or oligonucleotide that is essentially free from the remainder of the human genome and associated cellular or other impurities. This does not mean that the product has to have been extracted from the human genome; rather, the product could be a synthetic or cloned product for example.

As used herein, the term “nucleic acid”, also used in context as ‘target’ nucleic acid, may refer to any single- or double-stranded RNA or DNA molecule, such as mRNA, cDNA, and/or genomic DNA. The expression nucleic acid can be used interchangeably with oligo tag.

As used herein, “hybridizes” or “anneals” may mean that the primer or oligonucleotide forms a noncovalent interaction with the target nucleic acid molecule under standard stringency conditions. The hybridizing primer or oligonucleotide may contain non-hybridizing nucleotides that do not interfere with forming the noncovalent interaction, e.g., a 5′ tail or restriction enzyme recognition site to facilitate cloning.

Furthermore, as used herein, any “hybridization” may be performed under stringent conditions (e.g., high stringency or low stringency condition). The term “stringent conditions” may refer to any hybridization conditions which allow the primers to bind specifically to a nucleotide sequence within the allelic expansion, but not to any other nucleotide sequences. For example, specific hybridization of a probe to a nucleic acid target region under “stringent” hybridization conditions, include conditions such as 3× saline-sodium citrate (SSC), 0.1% sodium dodecyl sulfate (SDS), at 50° C. It is within the ambit of the skilled person to vary the parameters of temperature, probe length and salt concentration such that specific hybridization can be achieved. Hybridization and wash conditions are well known and exemplified in, for example, Sambrook, et al., Molecular Cloning: A Laboratory Manual, Second Edition, Cold Spring Harbor, N.Y., (1989), particularly Chapter 11 therein, incorporated herein by reference.

“Specific binding” or “specific hybridization”, with respect to nucleic acids, may mean that the primer forms a duplex (double-stranded nucleotide sequence) with the allelic expansion under the experimental conditions used, for example under stringent hybridization conditions, and that under those conditions the primer does not form a duplex with other regions of the nucleotide sequence present in the sample to be analyzed.

The nucleotide sequences presented herein can be contiguous, 5′ to 3′ nucleotide sequences, unless otherwise described.

The term “dNTPs” may refer to deoxyribonucleotide triphosphates comprising the four deoxyribonucleotides: dATP, dCTP, dGTP and dTTP, which can be polymerized by DNA polymerase to produce DNA. The deoxyribonucleotides may or may not be modified.

The term “chromatin” may refer to chromosomal DNA from eukaryotes, chromosomal DNA from prokaryotes, DNA from cellular organelles such as mitochondria and chloroplasts, and extra-chromosomal genetic elements such as plasmids and viral genomes. In one embodiment, chromatin may refer to a complex of nucleic acids and proteins in the cell nucleus that stains readily with basic dyes and condenses to form chromosomes during cell division. The term “chromatin fragments” may refer to chromatin that has been fragmented, for example by means of one or more of sonication, needle shear, nebulization, acoustic/mechanical shearing, point-sink shearing, passage through a French pressure cell, or enzymatic digestion with restriction endonucleases, DNase I, RNase, or similar methods.

The term “locus” (plural, “loci”) may refer to the location of a gene (or a significant sequence thereof) on a chromosome or on a linkage map. The term “genomic locus” may refer to the location of a gene in genomic DNA.

The term “bound” may refer to the binding of a protein, antigen or other small molecule to a nucleic acid or union of a protein, antigen or other small molecule with a nucleic acid, wherein the nucleic acid and protein, antigen or other small molecule are in covalent or non-covalent chemical combination/association with each other. For example a transcription factor can be “bound” directly to a nucleic acid. As another example, a first protein may be in a complex with a second protein, and the second protein may be attached to a nucleic acid. In this example, both proteins in the complex are “bound” to the nucleic acid.

A “biological sample” may refer to a sample of tissue and/or cells that has been obtained from, removed or isolated from a patient, animal, plant, or other organism, including eukaryotic or prokaryotic cells grown in culture or isolated from environmental samples.

The term “obtained” or “derived from” as used herein may be used inclusively. That is, it is intended to encompass any nucleotide sequence directly isolated from a biological sample or any nucleotide sequence derived from the sample.

As used herein, the term “aptamer” may refer to nucleic acids having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In one embodiment, the action is specific binding affinity for a target molecule, such target molecule being a three dimensional chemical structure other than a polynucleotide that binds to the aptamer through a mechanism which predominantly depends on Watson/Crick base pairing or triple helix binding, wherein the aptamer does not have the known physiological function of being bound by the target molecule.

The term “antigen binding protein” as used herein may refer to antibodies, antibody fragments and other protein constructs, such as domains, which can bind to an antigen.

The term “antibody” as used herein may refer to molecules with an immunoglobulin-like domain and includes monoclonal, recombinant, polyclonal, chimeric, humanized, bispecific and heteroconjugate antibodies; a single variable domain, a domain antibody, antigen binding fragments, immunologically effective fragments, single chain Fv, diabodies, TANDABS™, etc.

The term “non-Ig scaffold” may refer to a non-Ig domain that has been subjected to protein engineering in order to obtain binding to a ligand other than its natural ligand, for example a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

The term “peptide aptamer” may refer to combinatorial recognition molecules that contain a constant scaffold protein, typically thioredoxin (TrxA which contains a constrained variable peptide loop inserted at the active site.

The term “specifically binds”, as used throughout the present specification in relation to antigen binding proteins may mean that the antigen binding protein binds to a target epitope on IL-1b with a greater affinity than that which results when bound to a non-target epitope. In certain embodiments, specific binding refers to binding to a target with an affinity that is at least 10, 50, 100, 250, 500, or 1000 times greater than the affinity for a non-target epitope. For example, binding affinity may be as measured by routine methods, e.g., by competition ELISA or by measurement of equilibrium dissociation constant (Kd) with BIACORE™, KINEXA™ or PROTEON™.

The phrase “single variable domain” may refer to an antigen binding protein variable domain (for example, V_{H}, V_{HH}, V_{L}) that specifically binds an antigen or epitope independently of a different variable region or domain. A “domain antibody” or “dAb” may be considered the same as a “single variable domain” which can bing to an antigen. A single variable domain may be a human antibody variable domain, but also includes single antibody variable domains from other species such as rodent (for example, as disclosed in WO 00/29004), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “domain antibodies”. As used herein, V_{H} includes camelid V_{HH} domains.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain.

A “single variable domain” may be a folded polypeptide domain comprising sequences characteristic of antibody variable domains. It may include complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain can bind an antigen or epitope independently of a different variable region or domain.

An antigen binding fragment may be provided by means of arrangement of one or more CDRs on non-antibody protein scaffolds such as a domain. The domain may be a domain antibody or may be a domain which is a derivative of a scaffold selected from the group consisting of CTLA-4, lipocalin, SpA, an Affibody, an avimer, GroEl, transferrin, GroES and fibronectin/adnectin, which has been subjected to protein engineering in order to obtain binding to an antigen, other than the natural ligand.

An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, may mean +/−5% of the stated value, +/−4% of the stated value, +/−3% of the stated value, +/−2% of the stated value, +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Example

In the following example of the method described herein, proximity ligation was used to add double-stranded DNA (dsDNA) sequence tags preferentially to the ends of chromatin fragments that are reversibly crosslinked to a protein of interest.

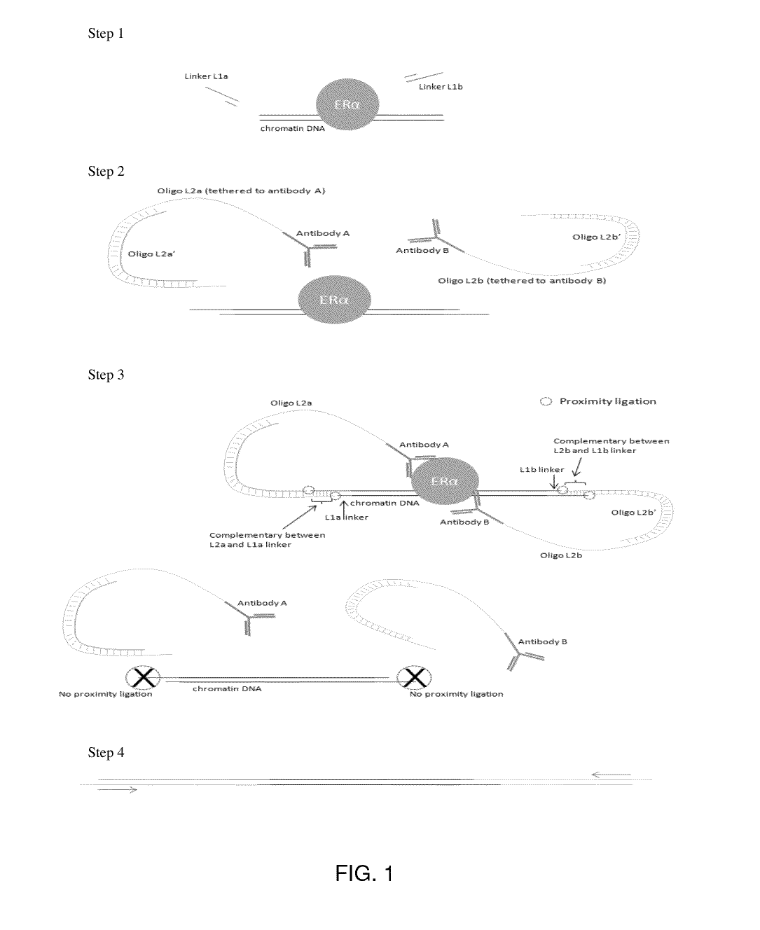

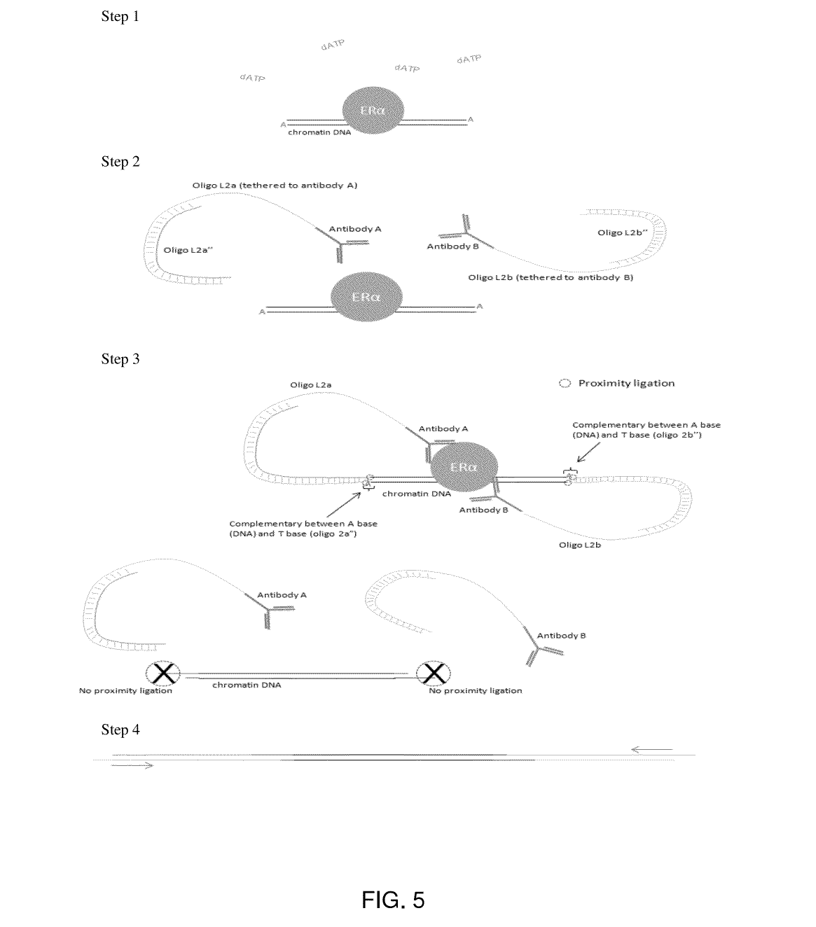

Cells were treated with formaldehyde and chromatin was purified and fragmented according to standard chromatin immunoprecipitation (ChIP) protocols. After repairing the DNA ends of the chromatin fragments to generate blunt-ends, the chromatin fragments were ligated to linkers containing 5′- or 3′-single-stranded DNA (ssDNA) overhangs, or were enzymatically treated to generate a 3′-deoxyadenine (dA) tail (the methods are illustrated in FIG. 1 and FIG. 5, respectively).

In particular, FIG. 1 shows a sample chromatin DNA fragment bound by a protein of interest which is recognized by antibody A and antibody B. Antibody A and antibody B are specifically tagged with oligonucleotides. Two different universal linkers (L1a and L1b) are ligated to the ends of the chromatin DNA fragment (Step 1). The oligonucleotide that is complementary to linker L1a is conjugated to antibody A and the oligonucleotide that is complementary to linker L1b is conjugated to antibody B (Step 2). The antibodies will specifically bind to the protein of interest. As both antibodies bind to the same protein, their respective oligonucleotides will be brought into spatial proximity with their complementary linkers. The complementary oligonucleotides and linkers then bind to each other (Step 3) to form a complex. The samples were subsequently diluted and briefly treated with DNA ligase to ligate the linkers and oligonucleotides (proximity dependent ligation). In control reactions, samples were incubated with equal concentrations of the free (unconjugated) antibodies and linkers, to measure the background rates of ligation in the absence of proximity effects. In the event that no protein is bound to the DNA, no proximity dependent ligation occurs (Step 3). After ligation, primers recognizing sequences on the oligonucleotides are used to specifically amplify the proximity-tagged chromatin DNA (Step 4).

FIG. 5 shows chromatin DNA fragments bound by a protein of interest which is recognized by antibody A and antibody B. Antibody A and antibody B are specifically tagged with oligonucleotides. The chromatin DNA is enzymatically treated to generate a 3′-dA tail (Step 1). The oligonucleotides of the antibodies are complementary to the 3′-dA tail are conjugated to antibody A and antibody B (Step 2). The antibodies will specifically bind to the protein of interest. As both antibodies bind to the same protein, their respective oligonucleotides will be brought into spatial proximity with the 3′-dA tails. The complementary oligonucleotides and the 3′-dA tails then bind to each other (Step 3) to form a complex. The complex is then incubated with a DNA ligase to ligate the linkers and oligonucleotides (proximity dependent ligation). In the event that no protein is bound to the DNA, no proximity dependent ligation occurs (Step 3). After ligation, primers recognizing sequences on the oligonucleotides are used to specifically amplify the proximity-tagged chromatin DNA (Step 4).

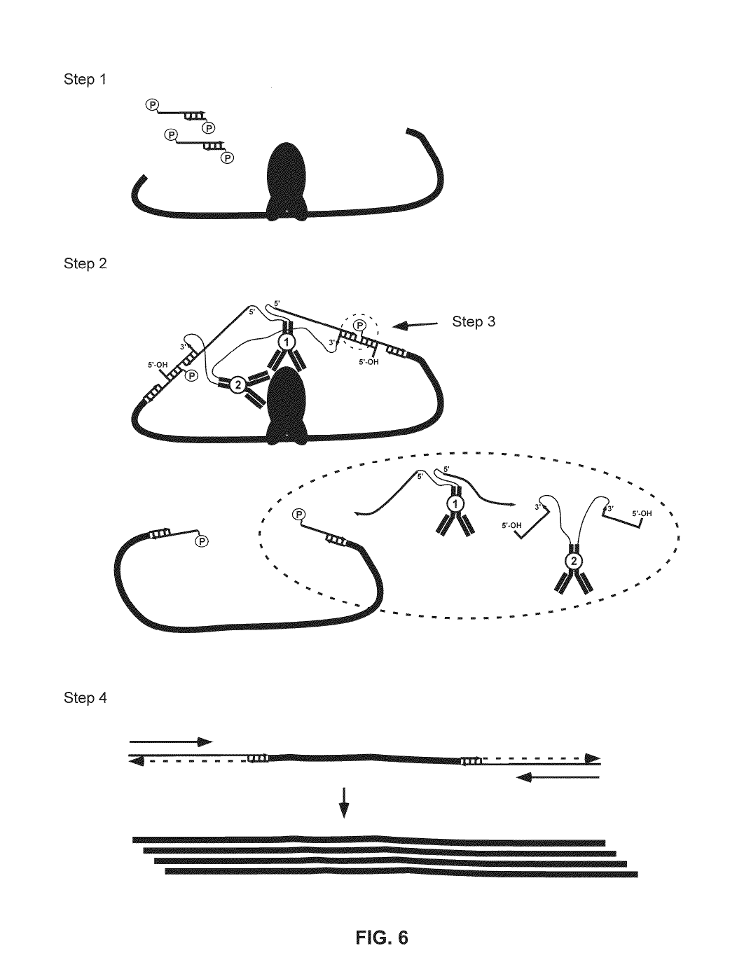

FIG. 6 shows another embodiment of the method as described herein. In Step 1, DNA linkers comprising a 5′ single stranded overhang are ligated to end repaired chromatin DNA fragments. The two linkers are provided with 5′ phosphates to prevent ligation to free 3′-OH ends. The linkers are non-complementary to prevent concatemers or the circularization of linker-ligated chromatin DNA fragments.

In Step 2, the chromatin DNA fragments are incubated with Antibody 1, which is tethered to a first and second oligonucleotide, and Antibody 2, which is tethered to a third and fourth oligonucleotide. Oligonucleotides three and four include adjacent sequences that are complementary to part of the first and second oligonucleotides and the single stranded DNA overhangs present on the linkers ligated to the chromatin DNA fragments. The formation of a complex between the first and third nucleic acid and linker at one end of the chromatin DNA fragment and a complex between the second and third nucleic acid and linker at the other end of the chromatin DNA fragment yields 2 nicked double stranded DNA portions which are substrates for a DNA ligase.

In Step 3, the nicked double-stranded DNA portions are incubated with a DNA ligase to ligate the first and second oligo tags or first and second nucleic acids to the linkers as long as first and second oligo tags or first and second nucleic acids are present in a ternary complex with the third and fourth oligonucleotides. In the event that no protein is bound to the DNA, no proximity dependent ligation occurs (Step 3)

In Step 4, chromatin DNA fragments flanked by the first and second oligo tags or first and second nucleic acids are specifically amplified by PCR using a primer that is complementary to a part of the first and second oligo tags or first and second nucleic acids.

All steps after chromatin fragmentation can be performed in a single EPPENDORF® tube.

The DNA binding specificity of a well-characterized transcription factor, estrogen receptor α (ERα), was assayed. The assay was sufficiently sensitive to map specific binding sites in as few as 5,000 cells. Thus, the method as described herein permits the enrichment of chromatin sequences bound by a protein of interest without the intervening immunoprecipitation and washing steps required in standard ChIP protocols.

### Material and Methods

### Cell Culture

Michigan Cancer Foundation-7 (MCF-7) cells (from a human breast adenocarcinoma cell line) were maintained in Minimal Essential Medium (MEM; Sigma) supplemented with 5% fetal calf serum (HyClone), 100 μg ml⁻¹ penicillin/streptomycin (Invitrogen), and 25 μg ml⁻¹ gentamicin (Invitrogen). Cells were grown in estrogen-depleted medium for at least 3 days prior to use in experiments. Estrogen-depleted medium was prepared by supplementing phenol red-free MEM (Sigma) with 5% heat inactivated calf serum [HyClone] that had been pre-treated with charcoal-dextran. Charcoal-dextran was prepared by incubating 0.25% (w/v) activated charcoal (Sigma) and 0.0025% (w/v) dextran (Sigma) in buffer (10 mM HEPES-KOH (4-(2-hydroxyethyl)-1-piperazineethanesulfonic acid—potassium hydroxide), pH 7.4, 250 mM sucrose, 1.5 mM MgCl₂) overnight at 4° C. For every 100 ml of serum to be treated, 100 ml of the charcoal-dextran slurry (corresponding to 253 mg charcoal-dextran) was centrifuged for 10 min at 500×g and the supernatant was discarded. Heat-inactivated calf serum (HyClone) was added to the charcoal-dextran pellet and incubated for 12 h at 4° C. Charcoal-dextran was then removed from the treated serum by centrifugation.

### Estrogen Treatment and Preparation of Chromatin

Estrogen-deprived MCF-7 cells were treated with 10 nM 17β-estradiol (E₂; Sigma) for 45 min. Following treatment, cells were cross-linked with 1.5% formaldehyde for 10 min, washed twice with ice-cold PBS, and harvested in ice-cold PBS plus 1× protease inhibitor cocktail (Roche). Cell pellets were resuspended in nuclei isolation buffer [50 mM Tris-Cl (pH 8.0), 60 mM KCl, 0.5% NP40, protease inhibitors (Roche Complete Protease Inhibitor Cocktail tablets)], centrifuged at 1,000×g for 3 min, and resuspended in lysis buffer [50 mM Tris-Cl (pH 8.0), 0.5% SDS, 10 mM EDTA, 0.5 mM EGTA, protease inhibitors]. Nuclei were sonicated 3× for 15 s using a Fisher Scientific Sonic Dismembrator Model 100 at 80% maximum power on ice to obtain chromatin fragments with an average size of 500-700 bp and then centrifuged at 14,000×g for 10 mins at 4° C. The supernatant was diluted with buffer [20 mM Tris-Cl (pH 8), 150 mM NaCl, 1% Triton X-100, 2 mM EDTA, protease inhibitors] to an equivalent of 5×10⁶ to 5×10⁷ cells ml⁻¹.

### Conjugation of Oligonucleotides to Antibodies

Oligonucleotides L2a and L2b were each synthesized a 3′ amino-modification and HPLC purified (Integrated DNA Technologies). The HC-20 antibody (Santa Cruz) was purified to remove gelatin prior to conjugation. Oligonucleotide L2a was conjugated to HC-20 antibody and oligonucleotide L2b was conjugated to Ab-10 antibody (Lab Vision; BSA-free preparation) using the Antibodies-Oligonucleotide Conjugation Kit (Solulink) following the manufacturer's protocol, which includes a clean-up step to remove unincorporated oligonucleotides. The oligonucleotides were then phosphorylated on their 5′ ends with T4 polynucleotide kinase (Fermentas) according to the enzyme supplier's recommended protocol (the enzyme was not heat inactivated after the reaction to avoid damaging the conjugated antibodies). In separate experiments, the oligonucleotides were synthesized with both a 5′ phosphate and a 3′ amino-modification followed by HPLC purification (Integrated DNA Technologies). To anneal complementary oligonucleotides, each antibody-conjugated oligonucleotides was added to an equimolar sample of the complementary oligonucleotide that had been pre-heated to 75° C. and the mixture was cooled slowly on the bench.

### Chromatin Enrichment by Antigen Proximity Tagging (CEAP)

End-Repair of Chromatin after Sonication.

Sheared chromatin (equivalent to 5×10⁵ cells; ˜3 μg genomic DNA) was incubated in 1× T4 DNA ligase buffer (Fermentas) with 0.5 mM ATP, 25 units of T4 polynucleotide kinase (Fermentas), 7.5 units of T4 DNA polymerase (NEB), 5 units of Klenow (NEB) and 0.4 mM dNTPs at 20° C. for 30 min followed by heat inactivation of the enzymes at 75° C. for 20 min. End-repaired chromatin samples were used directly in subsequent steps without further purification or cleanup.

Preparation of Chromatin for CEAP Using Universal Linkers.

Oligonucleotides L1a,

L1a′, L1b, and L1b′ (Table 1) were phosphorylated on their 5′ ends with T4 polynucleotide kinase (Fermentas) according to the enzyme supplier's recommended protocol followed by heat inactivation of the enzyme at 75° C. for 10 min. End-repaired chromatin was supplemented with 15 pmol linker L1a, 15 pmol linker L1b, 5% polyethylene glycol 4000 ( 1/10 dilution of 50% stock solution; Fermentas), and 25 units of T4 DNA ligase (Fermentas). Samples were incubated at 22° C. for 1 hour to ligate the linkers to the blunt-ended chromatin and then at 65° C. for 10 min to heat-inactivate T4 DNA ligase.

Preparation of Chromatin for CEAP Using dA-Tailing.

End-repaired chromatin was supplemented with 4 mM dATP and 15 units of Klenow exo-(NEB). Samples were incubated at 37° C. for 30 min and then at 75° C. for 20 min to heat-inactivate the enzyme.

Proximity ligation. For chromatin samples prepared with universal primers, 0.2 μg of HC-20 conjugated to L2a+L2a′ (Table 1) and 0.2 μg of Ab10 conjugated to L2b+L2b′ (Table 1) were added. In parallel control reactions, the corresponding amounts of the untethered antibodies and dsDNA oligonucleotides were added: 0.2 μg of HC-20, 0.2 μg of Ab-10, 0.68 pmol of oligonucleotides L2a+L2a′ and 1.69 pmol of oligonucleotides L2b+L2b′. For chromatin samples prepared by dA-tailing, the same additions were made except that primers L2a″ and L2b″ (Table 1) were used in place of L2a′ and L2b′ to create 3′-dT overhangs complementary to the dA-tailed chromatin ends. Following the addition of the antibodies and annealed oligonucleotides, the reaction volumes were bought up to 1 mL with 1× T4 DNA ligase buffer (Fermentas) and incubated at 4° C. overnight. The next morning, T4 DNA ligase (Fermentas) was added to each reaction (5 units for samples prepared with universal primers; 25 units for samples prepared by dA-tailing) and incubated at 22° C. for 10 min and then at 65° C. for 10 min to heat-inactivate DNA ligase. The reactions were incubated at 65° C. for at least 8 hours to reverse the formaldehyde crosslinks. DNA from each sample was then purified either by extraction with phenol/chloroform or using a QIAQUICK® PCR Purification Kit (Qiagen) prior to assaying samples by qPCR.

PCR Amplification of Ligation Products.

5 μl of each DNA sample was used as a template in PCR reactions containing 1×“Pfu buffer with MgSO₄” (Fermentas; 2 mM MgSO₄ final concentration), 2 units Pfu polymerase (Fermentas), 0.3 mM dNTPs, and 1.5 μM each of oligonucleotides PE1_L2 and PE2_L2 (Table 1). PCR was performed on a Bio-Rad Tetrad 2 using the thermal cycling parameters: 1) 95° C. for 3 min, 2) 15 cycles at 95° C. for 30 seconds, 65° C. for 30 seconds, 72° C. for 2 min, 3) a final extension at 72° C. for 10 min.

qPCR.

qPCR was performed using Maxima Sybergreen (Fermentas) and 350 nM of each primer (Table 2) on a Stratagene, MX3005p. The thermocycling parameters were: 1) 50° C. for 2 min (1 cycle); 2) 95° C. for 10 min (1 cycle); 3) 95° C. for 15 s followed by 60° C. for 60 s (40 cycles).

TABLE 1

| Oligo | Sequence (5′ - 3′) |
|---|---|
| L1a | # AGATCGGAAGAG (SEQ ID NO: 1) |
| L1a′ | # ACCGCTCTTCCGATCT (SEQ ID NO: 2) |
| L1b | # AGATCGGAAGAG (SEQ ID NO: 3) |
| | # GACGCTCTTCCGATCT (SEQ ID NO: 4) |
| L1b′ | # GACGCTCTTCCGATCT (SEQ ID NO: 5) |
| L2a | # CGGTTCAGCAGGAATGCCGAGACCGATCTCGTATGCCGTC |
| | TTCTGCTTGCTACCAGAGTC-3′-amino (SEQ ID NO: 6) |
| L2a′ | CAAGCAGAAGACGGCATACGAGATCGGTCTCGGCATTCCTGCTGA |
| | (SEQ ID NO: 7) |
| L2a″ | CAAGCAGAAGACGGCATACGAGATCGGTCTCGGCATTCCTGC |
| | TGAACCGT (SEQ ID NO: 8) |
| L2b | # CGTCGTGTAGGGAAAGAGTGTAGATCTCGGTGGTCGCCGTATC |
| | ATTTGACGTACAGTGTC-3′-amino (SEQ ID NO: 9) |
| L2b′ | AATGATACGGCGACCACCGAGATCTACACTCTTTCCCTACAC |
| | (SEQ ID NO: 10) |
| L2b″ | AATGATACGGCGACCACCGAGATCTACACTCTTTCCCTACACGAC |
| | GT (SEQ ID NO: 11) |
| PE1_L2 | CAAGCAGAAGACGGCATACGAGATCGGT (SEQ ID NO: 12) |
| PE2_L2 | AATGATACGGCGACCACCGAGATCT (SEQ ID NO: 13) |

5′ ssDNA overhangs of annealed linkers are underlined
# designates 5′ phosphorylation of oligonucleotide with Fermentas T4 polynucleotide kinase as per manufacturer's recommendation.

TABLE 2

| Oligo | Sequence (5′ - 3′) |
|---|---|
| Site 1 Fwd | CACCCCGTGAGCCACTGT |
| | (SEQ ID NO: 14) |
| Site 1 Rev | CTGCAGAAGTGATTCATAGTGAGAGAT |
| | (SEQ ID NO: 15) |
| Site 2 Fwd | GAGGTGTCTTGGCCACTGTT |
| | (SEQ ID NO: 16) |
| Site 2 Rev | GACTCCCACTGTCTCGAAGC |
| | (SEQ ID NO: 17) |
| Site 3 Fwd | TTGCTGTGCAAACAATAGCC |
| | (SEQ ID NO: 18) |
| Site 3 Rev | GTCCAAGGGCACATTCTCAT |
| | (SEQ ID NO: 19) |
| Site 4 Fwd | CGGTGGGTGCTAAAAGAAAC |
| | (SEQ ID NO: 20) |
| Site 4 Rev | TGGCTCACTCTTGCCTTTTT |
| | (SEQ ID NO: 21) |
| Site 5 Fwd | AGGAAGCAGGAAACCAAACA |
| | (SEQ ID NO: 22) |
| Site 5 Rev | CCCTGACCCACAAGGTCTTA |
| | (SEQ ID NO: 23) |
| Site 6 Fwd | AATACCTGAGGACCCCAACC |
| | (SEQ ID NO: 24) |
| Site 6 Rev | TCTTCACTCTCCTCGCATTG |
| | (SEQ ID NO: 25) |
| Site 7 Fwd | TTCACTCCCATTACCCAAGC |
| | (SEQ ID NO: 26) |
| Site 7 Rev | CCCAGCTACTCAGGAGGATG |
| | (SEQ ID NO: 27) |
| Site 8 Fwd | GGGACTCTCGAGGGGATAAG |
| | (SEQ ID NO: 28) |
| Site 8 Rev | TGTACCCAAGAACCACGTCA |
| | (SEQ ID NO: 29) |
| Site 9 Fwd | GAGGCTGTGCTTGGAGTAGG |
| | (SEQ ID NO: 30) |
| Site 9 Rev | CGTTTCCCCTGTGAAAGGTA |
| | (SEQ ID NO: 31) |
| Site 10 Fwd | TACCTCACTTGCCCACAACA |
| | (SEQ ID NO: 32) |
| Site 10 Rev | CCTCTCCTCCTGGCTTTTCT |
| | (SEQ ID NO: 33) |

### Results

The in vivo interactions between the transcription factor estrogen receptor alpha (ERα) and chromatin in the breast carcinoma cell line MCF-7 were analyzed. The binding sites of ERα in MCF-7 cells have been previously characterized by ChIP, providing an excellent reference data set.

Estrogen-deprived MCF-7 cells were treated with 17β-estradiol. The cells were then subjected to cross-linking using formaldehyde. The chromatin from the cells was then purified and fragmented by sonication. The chromatin fragments were end repaired and ligated with universal linkers L1a (the annealed oligonucleotides L1a and L1a′) and L1b (the annealed oligonucleotides L1b and L1b′). The method as described in FIG. 1 was then performed using an amount of chromatin equivalent to 50,000 cells.

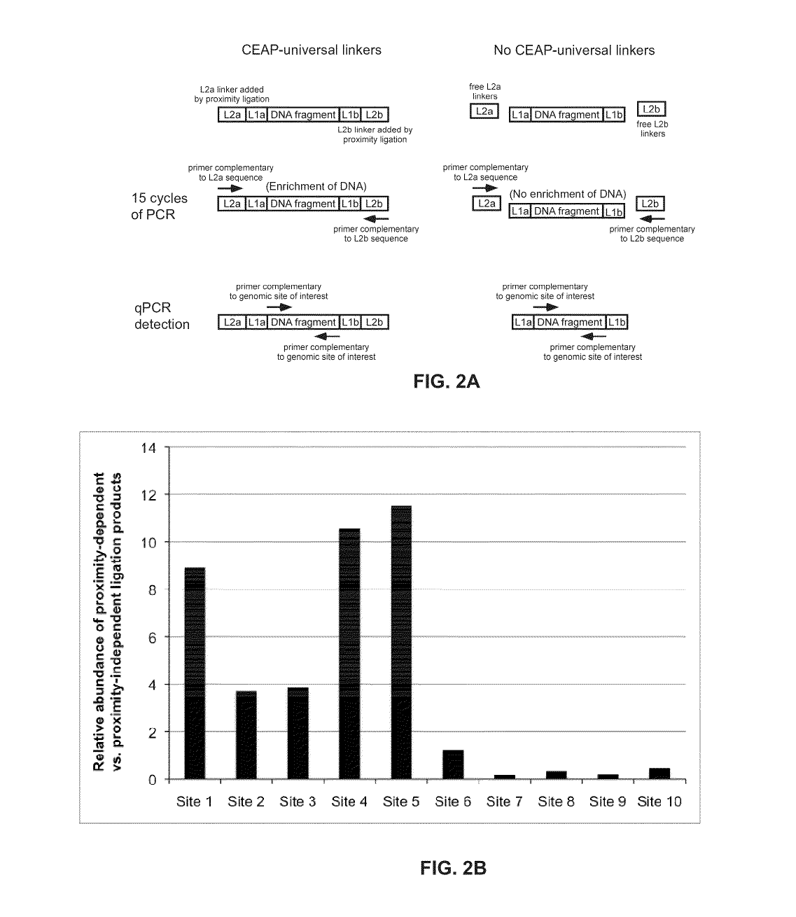

For the proximity ligation reactions, chromatin samples were incubated with two antibody-oligonucleotide conjugates: 1) the antibody HC-20 (Santa Cruz) conjugated to oligonucleotide L2a, which was annealed with oligonucleotide L2a′; 2) the antibody Ab-10 (Lab Vision) conjugated to oligonucleotide L2b, which was annealed with oligonucleotide L2b′. Both antibodies recognize ERα. In control reactions to measure the background levels of linker ligation in the absence of proximity effects, chromatin samples were incubated with equal amounts of the free (unconjugated) antibodies and linkers (HC-20, Ab10, L2a/L2a′, L2b/L2b′). The ligation reactions were performed under dilute conditions and for a limited period of time (10 min at 22° C.). Reactions were terminated by heat inactivating T4 DNA ligase at 65° C. for 10 min, and samples were then incubated for at least 8 hours at 65° C. to reverse the formaldehyde crosslinks. Chromatin fragments ligated to the L2a and L2b linkers were specifically amplified by 15 cycles of PCR using primers complementary to the linkers (oligonucleotides PE1_L2 and PE2_L; FIG. 2A).

The relative abundance of selected genomic sites in each of the samples was determined by qPCR and calculated the fold-enrichment of these sites in the proximity ligation samples (with antibody-conjugated linkers) compared to the control ligation samples (with unconjugated antibodies and linkers; FIG. 2B). Loci were selected to test based on previous studies mapping ERα binding sites in 17β-estradiol-treated MCF-7 cells. For the five sites tested where ERα had previously been reported to bind, proximity-dependent enrichment of 3- to 12-fold was observed (FIG. 2B, sites 1-5; see Table 2 for the primers used). In contrast, for the five sites tested where ERα binding had not been detected previously, little or no proximity-dependent enrichment was observed (FIG. 2B, sites 6-10). Thus, data obtained using the method as described herein were consistent with previous studies of ERα binding site specificity.

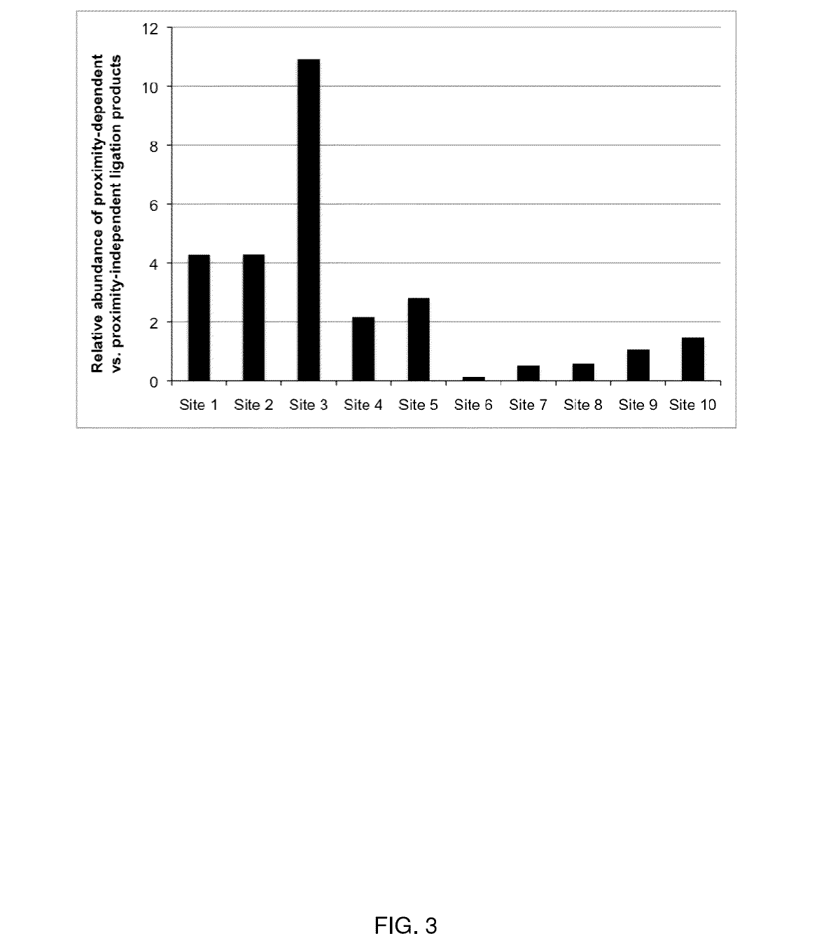

Another modified method as described herein in which dA tailing was used instead of linker ligation to prepare chromatin was followed (FIG. 5). The proximity ligation steps were performed as before, except that oligonucleotide L2a was annealed with oligonucleotide L2a″ and oligonucleotide L2b was annealed with oligonucleotide L2b″ to create 3′-dT overhangs complementary to the chromatin ends. Proximity-dependent enrichment of loci 1-5 after 15 cycles of PCR was observed. However, no enrichment of loci 6-10 was observed (FIG. 3).

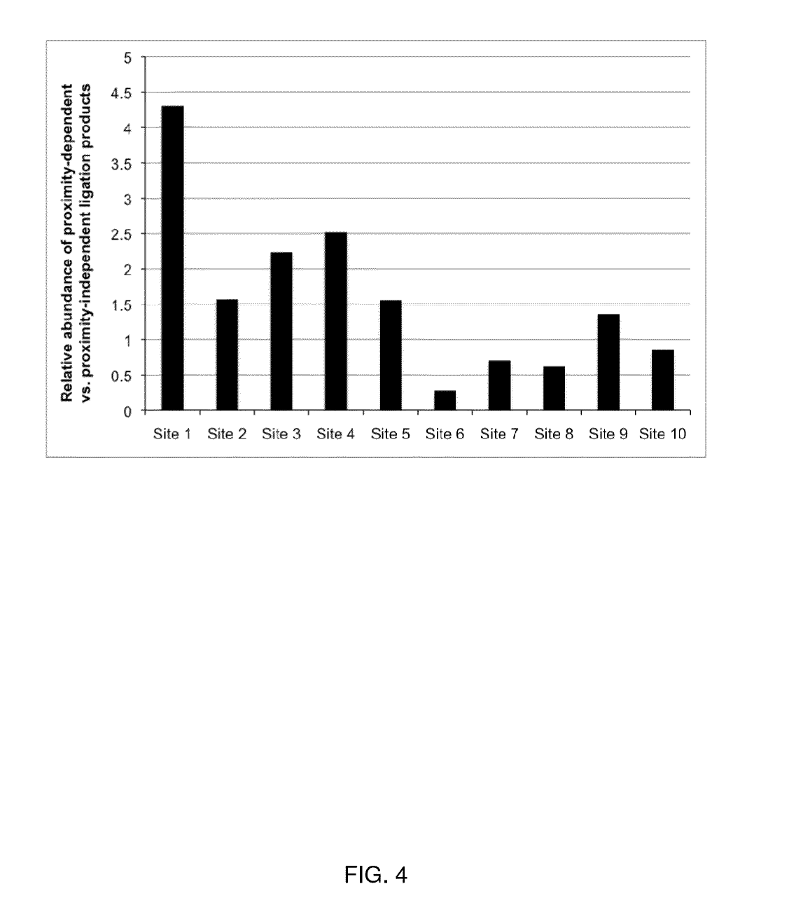

To examine the feasibility of the method described herein on low cell numbers, chromatin was isolated from 5000 17β-estradiol-treated MCF-7 cells. The method of the disclosure was performed on the isolated chromatin and similarly to the results above, enrichment of sites 1-5 was observed but not of sites 6-10 (FIG. 4). However, the fold-enrichment of sites 1-5 starting with 5,000 cells was smaller than the fold-enrichment observed starting with 50,000 cells (FIG. 2B).

We have shown that chromatin bound by the transcription factor ERα can be specifically enriched based on the proximity-dependent ligation of antibody-conjugated oligonucleotides to the ends of the chromatin fragments and that the technique is sufficiently sensitive to detect specific binding of ERα in as few as 5,000 cells. The method thus provides a highly sensitive alternative to conventional chromatin immunoprecipitation (ChIP).

By eliminating the requirement for mechanical steps such as centrifugation or magnetic capture to separate antibody-bound chromatin fragments from bulk DNA, the method can be performed in a single tube starting from fragmented chromatin DNA and is thus readily amenable to automation. The technique can be easily scaled to assay the binding of many different proteins in parallel, similar to the high-throughput assays for antigen detection that have been developed based on conventional proximity ligation assays.

Finally, using appropriately designed primers to PCR amplify proximity-dependent ligation products (Step 4 in FIGS. 1, 5 and 6), libraries compatible for Next Generation DNA sequencing can be prepared directly without further template preparation.

The method as described herein can determine the relative affinity of a protein for specific DNA sequences combined in a complex mixture within a single sample and is designed to assay protein binding to chromatin in living cells.

Additionally, the method as described herein may be designed to detect an antigen covalently attached to a fragment of chromatin DNA based on the proximity ligation of the DNA fragment to an oligonucleotide tethered to an antibody that recognizes the antigen.

The method as described herein can be used to study patient biopsied material or other samples where the amount of starting material is limiting.

The method as described herein can be used to enrich chromatin fragments that are covalently cross-linked to two different proteins by using antibody-oligonucleotide conjugates specific to each protein (a variation of the method illustrated in FIGS. 1, 5, and 6). Thus, it is possible to study, for example, the interactions between a transcription factor and its co-activator or co-repressor.

1. A method for identifying a target nucleic acid bound by an analyte in a sample comprising:
a. contacting said sample with a first probe comprising a first nucleic acid and a first analyte binding domain and a second probe comprising a second nucleic acid and second analyte binding domain, wherein said first and second probes can bind to said analyte such that said first and second nucleic acid are in spatial proximity to form a complex with said target nucleic acid if said target nucleic acid is bound by said analyte in said sample;
b. incubating said sample with a ligase that can ligate said complex to form a ligated target nucleic acid template;
c. amplifying said target nucleic acid template if present in said sample, and
d. detecting the presence or absence of an amplified target nucleic acid template.

2. A method for identifying a target nucleic acid bound by an analyte in a sample comprising:
a. contacting said sample with a first probe comprising a first and second nucleic acid and a first analyte binding domain; and a second probe comprising a third and fourth nucleic acid and a second analyte binding domain, wherein said second nucleic acid provides a region of complementarity for said third nucleic acid and one end of said target nucleic acid and said fourth nucleic acid provides a region of complementarity for said first nucleic acid and another end of said target nucleic acid, wherein said first and second probes can bind to said analyte, such that said first and second nucleic acids and said third and fourth nucleic acids are in spatial proximity to form a complex with said target nucleic acid if said target nucleic acid is bound by said analyte in said sample;
b. incubating said sample with a ligase that can ligate said complex to form a ligated nucleic acid template;
c. amplifying said target nucleic acid template;
d. detecting the presence or absence of an amplified nucleic acid template.

3. The method as claimed in claim 1, further comprising the step of cross-linking the analyte to the target nucleic acid in said sample with an agent that can cross-linking said analyte to said target nucleic acid prior to contacting the analyte with said first and second probes.

4. The method as claimed in claim 3, wherein said cross-linking agent is selected from formaldehyde.

5. The method as claimed in claim 1, further comprising the step of fragmenting the target nucleic acid prior to contacting the analyte with said first and second probes.

6. The method as claimed in claim 5, wherein said target nucleic acid is fragmented by sonication.

7. The method as claimed in claim 1, further comprising the step of modifying said target nucleic acid to form 3′ or 5′ single stranded nucleic acid overhangs at the ends of said target nucleic acid.

8. The method as claimed in claim 7, wherein the 3′ or 5′ single stranded nucleic acid overhangs are non-complementary.

9. The method as claimed in claim 1, wherein the analyte is a protein or a nucleic acid or a mixture thereof.

10. The method as claimed in claim 1, wherein the target nucleic acid is RNA or DNA.

11. The method as claimed in claim 10, wherein the target nucleic acid is DNA.

12. The method as claimed in claim 1, wherein the analyte binding domain comprises an aptamer, or antigen binding protein.

13. The method as claimed in claim 12, wherein the aptamer is an oligonucleotide or a peptide aptamer.

14. The method as claimed in claim 12, wherein the antigen binding protein is selected from the group consisting of antibodies, antibody fragments and other protein constructs.

15. The method as claimed in claim 14, wherein the antigen binding protein is an antibody.

16. The method as claimed in claim 15, wherein the antibody is selected from the group consisting of monoclonal, recombinant, polyclonal, chimeric, humanized, bispecific and heteroconjugate antibodies; a single variable domain, a domain antibody, antigen binding fragments, immunologically effective fragments, single chain Fv, diabodies and TANDABS™.

17. The method as claimed in claim 1, wherein the first analyte binding domain and said second analyte binding domain can specifically bind to the same analyte.

18. The method as claimed in claim 1, wherein the first analyte binding domain and said second analyte binding domain can specifically bind to different analytes.

19. The method as claimed in claim 1, wherein the ligating step comprises an enzymatic ligation reaction.

20-50. (canceled)

51. A kit for determining binding of an analyte to a target nucleic acid in a sample comprising:
a. a first container comprising a first probe, wherein said first probe comprises a first nucleic acid and a first analyte binding domain;
b. a second container comprising a second probe, wherein said second probe comprises a second nucleic acid and second analyte binding domain; and
c. instructions for using the first and second nucleic acid probes to detect an analyte in a sample.

52-107. (canceled)


SEQUENCE LISTING (33 sequences)

SEQ ID NO: 1 - DNA, 12 bp, Artificial Sequence
  Synthetic oligonucleotide
agatcggaagag

SEQ ID NO: 2 - DNA, 16 bp, Artificial Sequence
  Synthetic oligonucleotide
accgctcttccgatct

SEQ ID NO: 3 - DNA, 12 bp, Artificial Sequence
  Synthetic oligonucleotide
agatcggaagag

SEQ ID NO: 4 - DNA, 16 bp, Artificial Sequence
  Synthetic oligonucleotide
gacgctcttccgatct

SEQ ID NO: 5 - DNA, 16 bp, Artificial Sequence
  Synthetic oligonucleotide
gacgctcttccgatct

SEQ ID NO: 6 - DNA, 60 bp, Artificial Sequence
  Synthetic oligonucleotide
cggttcagcaggaatgccgagaccgatctcgtatgccgtcttctgcttgctaccagagtc

SEQ ID NO: 7 - DNA, 45 bp, Artificial Sequence
  Synthetic oligonucleotide
caagcagaagacggcatacgagatcggtctcggcattcctgctga

SEQ ID NO: 8 - DNA, 50 bp, Artificial Sequence
  Synthetic oligonucleotide
caagcagaagacggcatacgagatcggtctcggcattcctgctgaaccgt

SEQ ID NO: 9 - DNA, 60 bp, Artificial Sequence
  Synthetic oligonucleotide
cgtcgtgtagggaaagagtgtagatctcggtggtcgccgtatcatttgacgtacagtgtc

SEQ ID NO: 10 - DNA, 42 bp, Artificial Sequence
  Synthetic oligonucleotide
aatgatacggcgaccaccgagatctacactctttccctacac

SEQ ID NO: 11 - DNA, 47 bp, Artificial Sequence
  Synthetic oligonucleotide
aatgatacggcgaccaccgagatctacactctttccctacacgacgt

SEQ ID NO: 12 - DNA, 28 bp, Artificial Sequence
  Synthetic oligonucleotide
caagcagaagacggcatacgagatcggt

SEQ ID NO: 13 - DNA, 25 bp, Artificial Sequence
  Synthetic oligonucleotide
aatgatacggcgaccaccgagatct

SEQ ID NO: 14 - DNA, 18 bp, Artificial Sequence
  Synthetic oligonucleotide
caccccgtgagccactgt

SEQ ID NO: 15 - DNA, 27 bp, Artificial Sequence
  Synthetic oligonucleotide
ctgcagaagtgattcatagtgagagat

SEQ ID NO: 16 - DNA, 20 bp, Artificial Sequence
  Synthetic oligonucleotide
gaggtgtcttggccactgtt

SEQ ID NO: 17 - DNA, 20 bp, Artificial Sequence
  Synthetic oligonucleotide
gactcccactgtctcgaagc

SEQ ID NO: 18 - DNA, 20 bp, Artificial Sequence
  Synthetic oligonucleotide
ttgctgtgcaaacaatagcc

SEQ ID NO: 19 - DNA, 20 bp, Artificial Sequence
  Synthetic oligonucleotide
gtccaagggcacattctcat

SEQ ID NO: 20 - DNA, 20 bp, Artificial Sequence
  Synthetic oligonucleotide
cggtgggtgctaaaagaaac

SEQ ID NO: 21 - DNA, 20 bp, Artificial Sequence
  Synthetic oligonucleotide
tggctcactcttgccttttt

SEQ ID NO: 22 - DNA, 20 bp, Artificial Sequence
  Synthetic oligonucleotide
aggaagcaggaaaccaaaca

SEQ ID NO: 23 - DNA, 20 bp, Artificial Sequence
  Synthetic oligonucleotide
ccctgacccacaaggtctta

SEQ ID NO: 24 - DNA, 20 bp, Artificial Sequence
  Synthetic oligonucleotide
aatacctgaggaccccaacc

SEQ ID NO: 25 - DNA, 20 bp, Artificial Sequence
  Synthetic oligonucleotide
tcttcactctcctcgcattg

SEQ ID NO: 26 - DNA, 20 bp, Artificial Sequence
  Synthetic oligonucleotide
ttcactcccattacccaagc

SEQ ID NO: 27 - DNA, 20 bp, Artificial Sequence
  Synthetic oligonucleotide
cccagctactcaggaggatg

SEQ ID NO: 28 - DNA, 20 bp, Artificial Sequence
  Synthetic oligonucleotide
gggactctcgaggggataag

SEQ ID NO: 29 - DNA, 20 bp, Artificial Sequence
  Synthetic oligonucleotide
tgtacccaagaaccacgtca

SEQ ID NO: 30 - DNA, 20 bp, Artificial Sequence
  Synthetic oligonucleotide
gaggctgtgcttggagtagg

SEQ ID NO: 31 - DNA, 20 bp, Artificial Sequence
  Synthetic oligonucleotide
cgtttcccctgtgaaaggta

SEQ ID NO: 32 - DNA, 20 bp, Artificial Sequence
  Synthetic oligonucleotide
tacctcacttgcccacaaca

SEQ ID NO: 33 - DNA, 20 bp, Artificial Sequence
  Synthetic oligonucleotide
cctctcctcctggcttttct